(19) United States
(12) Patent Application Publication
(10) Pub. No.: US 2022/0071996 A1
(43) Pub. Date: Mar. 10, 2022

(54) ORAL FORMULATIONS OF BRANAPLAM

(71) Applicant: NOVARTIS AG, Basel (CH)

(72) Inventors: Manaud DE RASPIDE, Bottmingen (CH); Thomas FALLER, Reinach (CH); Claire HAUG, Saint-Louis (FR); Rohit LOWALEKAR, Telangana (IN); Paulo Antonio FERNANDES GOMES DOS SANTOS, Basel (CH)

(21) Appl. No.: 17/415,684

(22) PCT Filed: Dec. 18, 2019
(86) PCT No.: PCT/IB2019/061038

§ 371 Date: Jun. 17, 2021

(30) Foreign Application Priority Data
Dec. 21, 2018 (IN) 201811048667

(51) Int. Cl. A61K 31/501; A61K 47/26; A61K 47/46; A61K 47/40; A61P 21/00

(52) U.S. Cl. CPC A61K 31/501; A61K 47/26; A61P 21/00; A61K 47/40; A61K 47/46

(57) ABSTRACT

The present invention relates to pharmaceutical compositions suitable for oral administration comprising 5-(1H-pyrazol-4-yl)-2-(6-((2,2,6,6-tetramethylpiperidin-4-yl)oxy)pyridazin-3-yl)phenol (branaplam) and a pharmaceutically acceptable cyclodextrin.

### FIELD OF THE INVENTION

The present invention relates to paediatric pharmaceutical compositions suitable for oral administration comprising 5-(1H-pyrazol-4-yl)-2-(6-((2,2,6,6-tetramethylpiperidin-4-yl)oxy)pyridazin-3-yl)phenol (INN: branaplam) and a pharmaceutically acceptable cyclodextrin. In particular, the present invention relates to such compositions comprising hydroxypropyl-beta-cyclodextrin, one or more taste-enhancing/masking agents, and free of preservatives. The invention further provides methods of treating, preventing, or ameliorating a SMN-deficiency-related condition, comprising administering to a subject in need thereof an effective amount of the pharmaceutical composition disclosed herein.

### BACKGROUND

Proximal spinal muscular atrophy (SMA) is an inherited, clinically heterogeneous group of neuromuscular disorders characterized by degeneration of the anterior horn cells of the spinal cord. Patients suffer from symmetrical weakness of trunk and limb muscles, the legs being more affected than the arms and the proximal muscles weaker than the distal ones; diaphragm, facial and ocular muscles are spared. There are three forms of childhood-onset SMA (types I, II and III), and a relatively recently categorized adult-onset form IV, all of which can be distinguished on the basis of age of onset and severity of the clinical course assessed by clinical examination, muscle biopsy and electromyography (EMG) (Munsat T L, Davies K E (1992)).

Type I (Werdnig-Hoffmann disease) is the most acute and severe form, with onset before six months and death usually before two years; children are never able to sit without support.

Symptoms of the disease can be present in utero, as reduction of fetal movements; at birth; or more often, within the first four months of life. Affected children are particularly floppy, experience feeding difficulties and diaphragmatic breathing, and are characterized by a general weakness in the intercostals and accessory respiratory muscles. Affected children never sit or stand and usually die before the age of 2; death is generally due to respiratory insufficiency.

Type II (intermediate, chronic form) has onset between six and eighteen months of age; muscular fasciculations are common, and tendon reflexes progressively reduce. Children are unable to stand or walk without aid. Feeding and swallowing problems are not usually present in Type II SMA, although in some patients a feeding tube may become necessary. Most patients generally develop a progressive muscular scoliosis which can require surgical correction. Like patients with type I disease, clearing of tracheal secretions and coughing might become difficult because of poor bulbar function and weak intercostal muscles. These patients have profound hypotonia, symmetrical flaccid paralysis, and no control of head movement.

Type III (Kugelberg-Welander disease, or Juvenile Spinal Muscular Atrophy) is a mild, chronic form, with onset after the age of 18 months; motor milestones achievement is normal, and deambulation can be preserved until variable ages. These patients often develop scoliosis, and symptoms of joint overuse, generally caused by weakness, are frequently seen. Life expectancy is almost normal but quality of life is markedly compromised.

Types 1, II and III progress overtime, accompanied by deterioration of the patient's condition.

Adult-onset type IV is characterized by weakness in the second or third decade of life, with mild motor impairment not accompanied by respiratory or nutritional problems. Adult SMA is characterized by insidious onset and very slow progression. The bulbar muscles are rarely affected in Type IV. It is not clear that Type IV SMA is etiologically related to the Type I-III forms.

Other forms of spinal muscular atrophy include X-linked disease, spinal muscular atrophy with respiratory distress (SMARD), spinal and bulbar muscular atrophy (Kennedy's disease, or Bulbo-Spinal Muscular Atrophy), and distal spinal muscular atrophy.

SMA is due to mutations in the Survival of Motor Neuron (SMN) gene, which exists in two forms in humans (SMN1 and SMN2). Loss of SMN is deleterious to motor neurons and results in neuromuscular insufficiency, a hallmark of the disease. From a genetic point of view, SMA is an autosomal recessive condition, caused by disruption of SMN1 gene, located in 5q13 (Lefebvre S., et al. (1995) Cell 80: 155-165). More than 98% of patients with spinal muscular atrophy have a homozygous disruption of SMN1 by deletion, rearrangement, or mutation. All these patients, however, retain at least one copy of SMN2.

At the genomic level, only five nucleotides have been found that differentiate the SMN1 gene from the SMN2 gene. Furthermore, the two genes produce identical mRNAs, except for a silent nucleotide change in exon 7, i.e., a C→T change six base pairs inside exon 7 in SMN2. This mutation modulates the activity of an exon splicing enhancer (Lorson and Androphy (2000) Hum. Mol. Genet. 9:259-265). The result of this and the other nucleotide changes in the intronic and promoter regions is that most SMN2 are alternatively spliced, and their transcripts lack exons 3, 5, or 7. In contrast, the mRNA transcribed from the SMN1 gene is generally a full-length mRNA with only a small fraction of its transcripts spliced to remove exon 3, 5, or 7 (Gennarelli et al. (1995) Biochem. Biophys. Res. Commun. 213:342-348; Jong et al. (2000) J. Neurol. Sci. 173:147-153). All SMA subjects have at least one, and generally two to four copies of the SMN2 gene, which encodes the same protein as SMN1; however, the SMN2 gene produces only low levels of full-length SMN protein.

The SMNΔ7 protein is non-functional and thought to be rapidly degraded. About 10% of SMN2 pre-mRNA is properly spliced and subsequently translated into full-length SMN protein (FL-SMN), and the rest being the SMNΔ7 copy. The efficiency of SMN2 splicing might be dependent on severity of disease, and production of a full-length transcript of SMN2 could range from 10% to 50%. Furthermore, presence or absence of the SMN1 gene, roughly 90% of which becomes the FL-SMN gene product and protein, influences the severity of SMA by whether or not it can compensate for the truncated SMNΔ7 copies. A low level of SMN protein allows embryonic development, but is not sufficient to sustain the survival of motor neurons of the spinal cord.

The clinical severity of SMA patients inversely correlates with the number of SMN2 genes and with the level of functional SMN protein produced (Lorson C L, et al. (1999) PNAS; 96:6307-6311) (Vitali T. et al. (1999) Hum Mol Genet; 8:2525-2532) (Brahe C. (2000) Neuromusc. Disord.; 10:274-275) (Feldkotter M, et al. (2002) Am J Hum Genet; 70:358-368) (Lefebvre S, et al. (1997) Nature Genet; 16:265-269) (Coovert D D, et al. (1997) Hum Mol Genet; 6:1205-1214) (Patrizi A L, et al. (1999) Eur J Hum Genet; 7:301-309).

Current therapeutic strategies for SMA are mostly centered on elevating full length (wild type) SMN protein levels, modulating splicing towards exon 7 inclusion, stabilizing the wild type protein, and to a lesser extent, on restoring muscle function in SMA by providing trophic support or by inhibiting skeletal muscle atrophy.

The mechanism leading to motorneuron loss and to muscular atrophy still remains obscure, although the availability of animal models of the disease is rapidly increasing knowledge in this field (Frugier T, et al. (2000) Hum Mol. Genet. 9:849-58; Monani U R, et al. (2000) Hum Mol Genet 9:333-9; Hsieh-Li H M, et al. (2000) Nat Genet 24:66-70; Jablonka S, et al. (2000) Hum Mol. Genet. 9:341-6). Also the function of SMN protein is still partially unknown, and studies indicate that it can be involved in mRNA metabolism (Meister G, et al. (2002). Trends Cell Biol. 12:472-8; Pellizzoni L, et al. (2002). Science. 298: 1775-9), and probably in transport of proteins/mRNA to neuromuscular junctions (Ci-fuentes-Diaz C, et al. (2002) Hum Mol. Genet. 11: 1439-47; Chan Y B, et al. (2003) Hum Mol. Genet. 12:1367-76; McWhorter M L, et al. (2003) J. Cell Biol. 162:919-31; Rossoll W, et al. (2003) J. Cell Biol. 163:801-812).

In addition to the SMAs, a subclass of neurogenic-type arthrogryposis multiplex congenita (congenital AMC) has separately been reported to involve SMN1 gene deletion, suggesting that some degree of pathology in those afflicted is likely due to low levels of motor neuron SMN. (L. Burgien et al., (1996) J. Clin. Invest. 98(5):1130-32. Congenital AMC affects humans and animals, e.g., horses, cattle, sheep, goats, pigs, dogs, and cats. (M. Longeri et al., (2003) Genet. Sel. Evol. 35:S167-S175). Also, the risk of development or the severity of amyotrophic lateral sclerosis (ALS) has been found to be correlated with low levels of motor neuron SMN.

WO 2014/028459 discloses a group of SMA modulator compounds, in particular compounds to modulate SMN protein expression from SMN2 gene. Example 17-13 therein refers to branaplam in hydrochloride salt form. However, no particular formulation for branaplam is specified therein.

Branaplam is a small molecule with a molecular weight of 393.48. Branaplam is amphoteric, having measured pKas of 11.5 (acid), 9.8 (base) and 2.3 (base) and being of low lipophilicity with a measured log P of 2.6. Branaplam hydrochloride salt is crystalline with pH dependent solubility (e.g. in water), solubility decreasing upon increasing pH (solubility in pH 6.8 is 0.004 mg/mL). It is classified as a BCS class II molecule with low solubility (0.06 mg/mL in FeSSIF V2; 0.02 mg/mL in FaSSIF V2) and high permeability.

There is currently no available pharmaceutical formulation of branaplam suitable for paediatric use. Indeed, development of such formulation is substantially hampered by several technical challenges, such as poor solubility of branaplam in aqueous media (even in the presence of surfactants), pH-dependent stability (poor stability under pH 4), and incompatibility of branaplam with some preservatives such as potassium sorbate. In addition, the target population for such formulation, i.e. infants and children less than two years old, imposes further hurdles such as very limited choice of acceptable excipients, unpleasant taste of drug substance in oral solution, and the required dose flexibility and accuracy. Therefore, there is a strong need for development of an effective and suitable oral formulation of branaplam for paediatric use.

### BRIEF SUMMARY OF THE DISCLOSURE

The present invention provides an innovative paediatric oral solution to support the administration of branaplam, especially for patients less than two years old, providing dose flexibility, with good tolerability (preservative free) and no aftertaste combined with an aseptic manufacturing strategy.

In the first aspect, the present invention relates to a pharmaceutical composition comprising branaplam or a pharmaceutically acceptable salt thereof, and a pharmaceutically acceptable cyclodextrin or combination of pharmaceutically acceptable cyclodextrins.

In a second aspect, the present invention relates to the pharmaceutical composition of the first aspect for use in treating, preventing or ameliorating a SMN-deficiency-related condition.

In a third aspect, the present invention relates to a method for treating, preventing or ameliorating a SMN-deficiency-related condition, comprising administering to a subject in need thereof an effective amount of the pharmaceutical composition of the first aspect.

In a fourth aspect, the present invention relates to the pharmaceutical composition of the first aspect for the manufacture of a medicament for the treatment or prevention or amelioration of a SMN-deficiency-related condition.

### BRIEF DESCRIPTION OF THE DRAWINGS

FIG. 1 shows the process flow diagram for preparation of branaplam solution exemplified in Example 25a.

### DEFINITIONS

In order that the present disclosure may be more readily understood, certain terms are first defined. Additional definitions are set forth throughout the detailed description.

As used herein, the term “a”, “an”, “the” and similar terms used in the context of the present disclosure (especially in the context of the claims) are to be construed to cover both the singular and plural unless otherwise indicated herein or clearly contradicted by the context. As such, the terms “a” (or “an”), “one or more”, and “at least one” can be used interchangeably herein.

“And/or” means that each one or both or all of the components or features of a list are possible variants, especially two or more thereof in an alternative or cumulative way.

The term “about” in relation to a numerical value X means, for example, X±15%, including all the values within this range.

As used herein, the terms “free form” or “free forms” or “in free form” or “in the free form” refers to the compound in non-salt form, such as the base free form.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

The term “pharmaceutical composition” is defined herein to refer to a mixture or solution containing at least one therapeutic agent to be administered to a subject, e.g., a human, in order to prevent or treat a particular disease or condition affecting the human.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “patient” or “subject” are taken to mean a human. Except when noted, the terms “patient” or “subject” are used herein interchangeably.

As used herein, a subject is “in need of” a treatment if such subject would benefit biologically, medically or in quality of life from such treatment.

The term “once a week” or “once weekly” in the context of administering a drug means herein administering one dose of a drug once each week, wherein the dose is, for example, administered on the same day of the week.

The term “twice a week” in the context of administering a drug means herein administering one dose of a drug twice each week, wherein each administration is, for example, on separate days, for example, at regular intervals of, for example, 72 hours.

The terms “drug”, “active substance”, “active ingredient”, “pharmaceutically active ingredient”, “active agent”, “therapeutic agent” or “agent” are to be understood as meaning a compound in free form or in the form of a pharmaceutically acceptable salt. In particular, in the context of the present invention, it is branaplam or a pharmaceutically acceptable salt thereof.

A formulation of the invention will comprise an active agent present in an effective amount. By the term “effective amount” or “therapeutically effective amount” or “pharmaceutically effective amount”, is meant the amount or quantity of active agent that is sufficient to elicit the required or desired response, or in other words, the amount that is sufficient to elicit an appreciable biological response when administered to a subject. It is understood that an “effective amount” or a “therapeutically effective amount” can vary from subject to subject, due to variation in metabolism of branaplam, age, weight, general condition of the subject, the condition being treated, the severity of the condition being treated, and the judgment of the prescribing physician.

The term “treatment” includes: (1) preventing or delaying the appearance of clinical symptoms of the state, disorder or condition developing in an animal, particularly a mammal and especially a human that may be afflicted with or predisposed to the state, disorder or condition but does not yet experience or display clinical or subclinical symptoms of the state, disorder or condition; (2) inhibiting the state, disorder or condition (e.g. arresting, reducing or delaying the development of the disease or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be an effective treatment.

As used herein, the term “SMN-deficiency-related conditions” includes but is not limited to Spinal Muscular Atrophy (SMA), neurogenic-type arthrogryposis multiplex congenita (congenital AMC), and amyotrophic lateral sclerosis (ALS).

As used herein, the term “Spinal Muscular Atrophy”, or “SMA,” include three forms of childhood-onset SMA: Type I (Werdnig-Hoffmann disease); Type II (intermediate, chronic form), Type III (Kugelberg-Welander disease, or Juvenile Spinal Muscular Atrophy); Adult-onset type IV; as well as other forms of SMA, including X-linked disease, spinal muscular atrophy with respiratory distress (SMARD), spinal and bulbar muscular atrophy (Kennedy's disease, or Bulbo-Spinal Muscular Atrophy), and distal spinal muscular atrophy.

As used herein, the term “inhibit”, “inhibition” or “inhibiting” refers to the reduction or suppression of a given condition, symptom, or disorder, or disease, or a significant decrease in the baseline activity of a biological activity or process.

### DETAILED DESCRIPTION

During development of an oral formulation of branaplam, it was found that the solubility of branaplam is very low in water. However, surprisingly, with the use of a special solubilizer, sufficient solubility of branaplam can be achieved.

During the manufacturing procedure, it was found that potassium sorbate (a preservative) was not compatible or efficient in the developed formulation. As a result, an acceptable paraben free preserved paediatric formulation could not be made. However, this is overcome in the context of the present invention by using an aseptic manufacturing procedure, optionally supplemented by the use of appropriate bottles with child-resistant/tamper evident caps.

Therefore, the present invention is based on the surprising finding that using a special composition and manufacturing procedure, it is possible to develop a stable formulation of branaplam suitable for paediatric use. This formulation overcomes the pH dependent solubility of branaplam (e.g. in water), where solubility decreases upon increasing pH (solubility of branaplam hydrochloride salt in pH 6.8 is 0.004 mg/mL), enabling a concentration of 1 mg/ml or higher, adapted to the medication intended use; this formulation and the excipients therein also support the special target population (<2 years and very sick); Being a single-use preservative-free formulation, it avoids 1) high preservative level burden due to the interaction of the HP-b-CD (hydroxylpropyl cyclodextrine) with the preservatives, 2) chemical interaction of branaplam with potassium sorbate, as well as 3) limited number of approved preservatives suitable for infants (<2 years old). The disclosed formulation also has a suitable aftertaste, overcoming the aversive taste of branaplam, and can be produced by an aseptic manufacturing process to support manufacturing of a preservative-free formulation.

Formulations can also be used in adult populations to treat SMA type II and III.

In the first aspect, the present invention is related to a pharmaceutical composition comprising branaplam or a pharmaceutically acceptable salt thereof, and a pharmaceutically acceptable cyclodextrin or combination of pharmaceutically acceptable cyclodextrins.

Branaplam is the INN of 5-(1H-pyrazol-4-yl)-2-(6-((2,2,6,6-tetramethylpiperidin-4-yl)oxy)pyridazin-3-yl)phenol and is characterized by the following chemical formula (I):

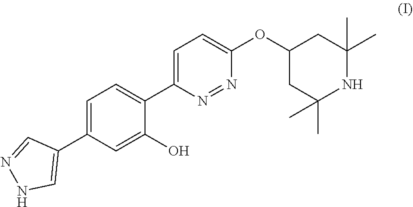 [SMILES: CC1(C)CC(Oc2ccc(-c3ccc(-c4cn[nH]c4)cc3O)nn2)CC(C)(C)N1]

The present application includes pharmaceutically acceptable salts (preferably derived from inorganic or organic acids), solvates, hydrates, enantiomers, polymorphs or mixtures thereof of branaplam.

“Branaplam” or “branaplam free base” or “branaplam base” or “branaplam in the free form” or “branaplam in free form” refers to the compound of formula (I), as herein, in the free form, and any reference to “a pharmaceutically acceptable salt thereof” refers, in particular, to a pharmaceutically acceptable acid addition salt thereof. In a preferred embodiment, the term “branaplam, or a salt thereof, such as a pharmaceutically acceptable salt thereof”, as used in the context of the present invention (especially in the context of any of the embodiments, above or below, and the claims) is thus to be construed to cover both the compound of formula (I), as herein, in the free form and a pharmaceutically acceptable salt thereof, unless otherwise indicated herein. As used herein, the term “branaplam hydrochloride salt” or “branaplam monohydrochloride salt” or “branaplam in hydrochloride salt form” refers to 5-(1H-Pyrazol-4-yl)-2-(6-((2,2,6,6-tetramethylpiperidin-4-yl)oxy)pyridazin-3-yl)phenol hydrochloride salt. In particular, branaplam is in the form of the hydrochloride salt. Branaplam, or a pharmaceutical salt thereof, such as branaplam hydrochloride salt, can be prepared as described in WO2014/028459 (e.g. in Example 17-13), which is incorporated herein by reference.

As used herein, reference to an amount of branaplam (e.g. mg, percentage) is to be understood to be the amount of the compound of formula (I), as herein, in the free form. As used herein, reference to an amount of branaplam, or a pharmaceutical acceptable salt thereof, (e.g. mg, percentage), is to be understood to the amount of the compound of formula (I), as herein, in the free form, which will be adapted accordingly for a pharmaceutically acceptable salt.

As used herein, reference to a concentration of branaplam (e.g. mg/ml) is to be understood to the amount of the compound of formula (I), as herein, in the free form. As used herein, reference to an concentration of branaplam, or a pharmaceutical acceptable salt thereof, (e.g. mg/ml), is to be understood to the amount of the compound of formula (I), as herein, in the free form, which will be adapted accordingly for a pharmaceutically acceptable salt.

As used herein, “pharmaceutically acceptable salt” refers to derivatives of the disclosed compounds wherein the active agent is modified by reacting it with an acid or base as needed to form an ionically bound pair. Pharmaceutically acceptable salts retain the biological effectiveness and properties of the compound and typically are not biologically or otherwise undesirable. The compounds of the present disclosure that are basic in nature are capable of forming a wide variety of salts with various inorganic and organic acids. The acids that may be used to prepare pharmaceutically acceptable acid addition salts of such basic compounds of the present disclosure are those that form non-toxic acid addition salts, i.e., salts containing pharmaceutically acceptable anions, such as the acetate, adipate, alginate, aspartate, benzoate, benzenesulfonate, besylate, bisulfate, butyrate, bromide/hydrobromide, bicarbonate/carbonate, bisulfate/sulfate, camphorsulfornate, camphorate, chloride/hydrochloride, chlortheophyllonate, citrate, cyclopentanepropionate, digluconate, dodecylsulfate, ethandisulfonate, ethanesulfonate, fumarate, gluceptate, glucoheptanoate, glycerophosphate, gluconate, glucuronate, glycolate, hemisulfate, heptanoate, hexanoate, hippurate, hydroiodide/iodide, isethionate, lactate, lactobionate, laurylsulfate, malate, maleate, malonate, mandelate, mesylate, methanesulfonate, methylsulphate, naphthoate, napsylate, nicotinate, nitrate, octadecanoate, oleate, oxalate, palmitate, pamoate, picrate, pivalate, phosphate/hydrogen phosphate/dihydrogen phosphate, polygalacturonate, propionate, salicylate, stearate, succinate, sulfate, sulfosalicylate, tartrate, thiocyanate, toluenesulfonate, tosylate, trifluoroacetate, undecanoate, 2-hydroxy-ethanesulfonate and 2-naphthalenesulfonate. Lists of other suitable salts can be found, e.g., in “Remington's Pharmaceutical Sciences”, 20^{th} ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002). Since a single compound of the present disclosure may include more than one acidic or basic moieties, the compounds of the present disclosure may include mono-, di- or tri-salts in a single compound.

Branaplam is a small molecule with a molecular weight of 393.48. This molecule is amphoteric having measured pKas of 11.5 (acid), 9.8 (base) and 2.3 (base) and being of low lipophilicity with a measured log P of 2.6 and pH dependent solubility (e.g. in water), solubility decreasing upon increasing pH (solubility of branaplam hydrochloride salt in pH 6.8 is 0.004 mg/mL). On the other hand, the use of solubilizers for administration to an infant is limited to a few excipients and at limited concentrations. The following formulation strategies adapted to paediatric target population were investigated in order to increase the solubility of the drug and unlock the branaplam therapeutic benefit to patients: use of cosolvent (e.g., PEG 300, PEG 400, Glycerol, Propylene glycol, etc.); use of surfactants (e.g., Cremophor RH 40, Tween 80, etc.); and pH adjustment. Surprisingly, only beta-cyclodextrin-based vehicles were found to enhance branaplam solubility to the required levels, and up to ˜10 mg/mL of branaplam (e.g. up to about 10 mg/mL of branaplam) could be dissolved at room temperature using 17.5% HP-b-CD. Not only the solubility could be increased, but the formulation could also be effectively diluted with a wide range of vehicles including milk (i.e., Aptamil®, Bimbosam®, Similac®) with good chemical stability and without precipitation.

Cyclodextrins (also known as cycloamyloses) are a family of cyclic oligomers composed of α-(1→4)-linked D-glucopyranose units in 4C₁ chair conformation. The three common α-, β-, and γ-cyclodextrins consist of six, seven, and eight D-glucopyranose units, respectively. The cyclodextrins may be pictured as hollow truncated cones with hydrophilic exterior surfaces and hydrophobic interior cavities. In aqueous solutions, these hydrophobic cavities provide a haven for hydrophobic organic compounds that can fit all or part of their structure into these cavities. This process, known as inclusion complexation, may result in increased apparent aqueous solubility and stability for the complexed drug. The complex is stabilized by hydrophobic interactions and does not involve the formation of any covalent bonds.

Due to the chair conformation of the glucopyranose units, the cyclodextrins are shaped like a truncated cone rather than perfect cylinders. The hydroxyl functions are orientated to the cone exterior with the primary hydroxyl groups of the sugar residues at the narrow edge of the cone and the secondary hydroxyl groups at the wider edge. The central cavity is lined by the skeletal carbons and ethereal oxygens of the glucose residues, which gives it a lipophilic character.

The chemical structure (left) and the toroidal shape (right) of the β-cyclodextrin molecule are illustrated in Scheme 1 below.

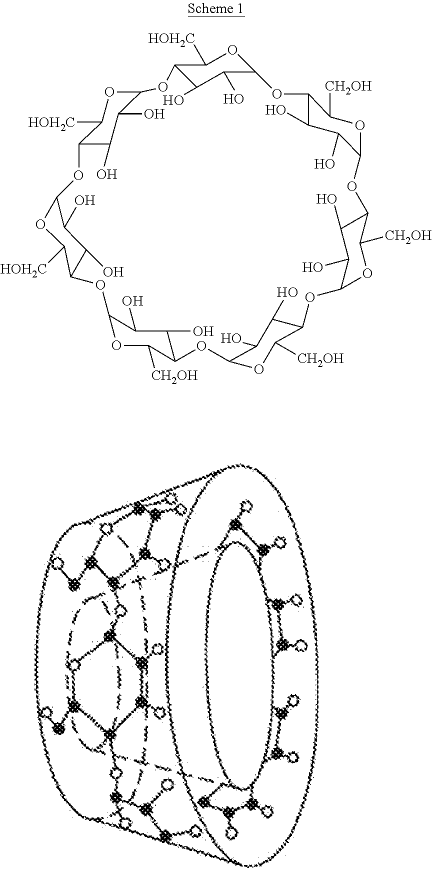 [SMILES: OCC1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O]

The natural cyclodextrins, in particular β-cyclodextrin, are of limited aqueous solubility meaning that complexes resulting from interaction of lipophiles with these cyclodextrin can be of limited solubility, resulting in precipitation of solid cyclodextrin complexes from water and other aqueous systems. In fact, the aqueous solubility of the natural cyclodextrins is much lower than that of comparable acyclic saccharides. This is thought to be due to relatively strong intermolecular hydrogen bonding in the crystal state. Substitution of any of the hydrogen bond forming hydroxyl groups, even by lipophilic methoxy functions, results in dramatic improvement in their aqueous solubility. Cyclodextrin derivatives of pharmaceutical interest include the hydroxypropyl derivatives of β- and γ-cyclodextrin, the randomly methylated β-cyclodextrin, sulfobutylether β-cyclodextrin, and the so-called branched cyclodextrins such as glucosyl-β-cyclodextrin. Structure and solubility of β-cyclodextrin and some of its derivatives are presented below.

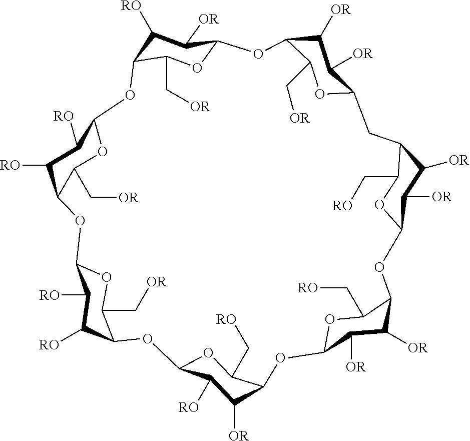

| | | Solubility in |
|---|---|---|
| Cyclodextrin | R | water (mg/ml) |
| β-cyclodextrin | —H | 18.5 |
| 2-Hydroxypropyl β- | —CH₂CHOHCH₃ | >600 |
| cyclodextrin | | |
| Sulfobutylether β- | —(CH₂)₄SO₃⁻Na⁺ | >500 |
| cyclodextrin sodium | | |
| salt | | |

The natural α- and β-cyclodextrin, unlike γ-cyclodextrin, cannot be hydrolyzed by human salivary and pancreatic amylases. However, both α- and β-cyclodextrin can be fermented by the intestinal microflora. Cyclodextrins are both large (MW ranging from almost 1000 to over 2000 Daltons) and hydrophilic with a significant number of H-donors and acceptors and, thus, are not absorbed from the gastrointestinal tract in their intact form. Hydrophilic cyclodextrins are considered non-toxic at low to moderate oral dosages. Lipophilic cyclodextrin derivatives, such as the methylated cyclodextrins, are to some extent absorbed from the gastrointestinal tract in to the systemic circulation and have been shown to be toxic after parenteral administration.

In one embodiment, the pharmaceutically acceptable cyclodextrin is a beta-cyclodextrin. More preferably, the beta-cyclodextrin is chemically modified, especially alkylated or hydroxyl-alkylated. Non-limiting examples of suitable modified beta-cyclodextrins are 2-hydroxypropyl-beta-cyclodextrin (also known as Hydroxypropyl Betadex; denoted by HP-b-CD), sulfobutylether-beta-cyclodextrin or its sodium salt (also known as Betadex Sulfobutyl Ether Sodium and sodium sulfobutylether beta-cyclodextrin; denoted by SBE-b-CD; commercially available under the brand name Captisol® by CyDex Pharmaceuticals, Inc.), 6-O-p-toluenesulfonyl-beta-cyclodextrin, beta-cyclodextrin, beta-cyclodextrin phosphate sodium salt, beta-cyclodextrin sulphate sodium salt, butyl-beta-cyclodextrin, carboxymethyl-beta-cyclodextrin sodium salt, diglucosyl beta-cyclodextrin, dihydroxypropyl-beta-cyclodextrin, dimaltosyl-beta-cyclodextrin, dimethyl beta-cyclodextrin, ethyl-beta-cyclodextrin, glucosyl-beta-cyclodextrin, heptakis (2,3,6-tri-O-benzoyl)-beta-cyclodextrin, heptakis (2,3,6-tri-O-methyl)-beta-cyclodextrin, heptakis (6-O-sulfo)-beta-cyclodextrin heptasodium salt, hydroxyethyl-beta-cyclodextrin, hydroxypropyl-beta-cyclodextrin, maltosyl-beta-cyclodextrin, maltotriosyl-beta-cyclodextrin, methyl-beta-cyclodextrin, Succinyl-(2-hydroxypropyl)-beta-cyclodextrin, triacetyl-beta-cyclodextrin, and mixtures thereof such as maltosyl-beta-cyclodextrinldimaltosyl-beta-cyclodextrin, as well as methyl-beta-cyclodextrin. Alkylcyclodextrins (e.g. methyl dimethyl-cyclodextrin, diethyl-cyclodextrin), carboxyalkylcyclodextrins (e.g. carboxymethyl-cyclodextrin) and the like. In a more preferred embodiment, said chemically-modified beta-cyclodextrin is 2-hydroxypropyl beta-cyclodextrin. Procedures for preparing such cyclodextrin derivatives are well known, for example, from Bodor U.S. Pat. No. 5,024,998 dated Jun. 18, 1991, and references cited therein.

In case of hydroxypropylated beta-cyclodextrin, the average degree of substitution preferably varies from 2.8 to 10.5, more preferably from 4 to 8, even more preferably from 5.5 to 6.9, in particular from about 6.1 to about 6.3. The average degree of substitution is understood as number of substituents per cyclodextrin ring. Especially an average degree of substitution of 5 to 7, in particular of about 6.2, leads to superior dissolution properties.

In the process for producing the branaplam formulation according to the present invention, the molar ratio of cyclodextrin to branaplam is usually 1:4 to 200:1, preferably from 1:2 to 100:1, more preferably from 1:1 to 50:1, even more preferably from 2:1 to 25:1, or from 2:1 to 20:1, or from 3:1 to 15:1, in particular about 13.2:1.

The compounds and salts of the composition of the present invention encompass hydrate and solvate forms.

In one embodiment, the pharmaceutical composition is a liquid composition. Preferably, said composition is a solution. In a preferred embodiment, the solvent is water.

In one embodiment, the composition of the present invention is in the form of a concentrate. Within this application, a “concentrate” is referred to as a formulation which preferably is not administered directly to a patient but diluted before use. For example, the concentrate can be diluted with a suitable liquid, e.g. water, alternatively with 5% glucose solutions or saline, to give a ready-for-use formulation. Alternatively, the concentrate may be used directly.

In one embodiment, the concentration of branaplam or any pharmaceutically acceptable salt thereof is in the range from about 1 mg/ml to about 30 mg/ml. In a preferred embodiment, said concentration is in the range from about 3 mg/ml to about 10 mg/ml. In a more preferred embodiment, said concentration is about 3.5 mg/ml. In particular, amounts (i.e. mg/ml) refer to an amount of branaplam [i.e. compound of formula (I), as herein, in the free form], and if a salt thereof (e.g., hydrochloride salt) is used, the amount will be adapted accordingly.

In one embodiment, the concentration of cyclodextrin is in the range of 0.1 percent to 70 percent (w/v). In a preferred embodiment, said concentration is in the range of 2 percent to 25 percent (w/v). In another preferred embodiment, said concentration is in the range of 2 percent to 20 percent (w/v). In a more preferred embodiment, said concentration is about 17.5 percent (w/v).

In one embodiment, the pH of the composition is in the range of 3.5-9. In a preferred embodiment, the pH of the composition is about 4. In another embodiment, the pH of the composition is in the range of 3.5 to 7 or in the range of 4 to 7.

In a preferred embodiment, the pharmaceutical composition comprises branaplam or a pharmaceutically acceptable salt thereof in a concentration of 1 mg/ml to 30 mg/ml, 2-hydroxypropyl-beta-cyclodextrin in a concentration in the range of 2 percent to 20 percent (w/v), and the pH of the composition is about 4. In particular, amounts (i.e. mg/ml) refer to an amount of branaplam [i.e. compound of formula (I), as herein, in the free form], and if a salt thereof (e.g., hydrochloride salt) is used, the amount will be adapted accordingly.

In a preferred embodiment, the pharmaceutical composition comprises branaplam monohydrochloride salt in a concentration of 1 mg/ml to 30 mg/ml, 2-hydroxypropyl-beta-cyclodextrin in a concentration in the range of 0.1 percent to 70 percent (w/v), and the pH of the composition is in the range of 3.5 to 9, wherein pH is adjusted by using acids (e.g., hydrochloride acid, acetic acid, phosphoric acid, lactic acid, tartaric acid, citric acid), or bases (e.g. sodium hydroxide).

In a preferred embodiment, the pharmaceutical composition comprises branaplam monohydrochloride salt in a concentration of 1 mg/ml to 40 mg/ml, sulfobutylether β-cyclodextrin sodium salt (e.g. Captisol®) in a concentration in the range of 0.1 percent to 70 percent (w/v), and the pH of the composition is in the range of 3.5 to 9, wherein pH is adjusted by using acids (e.g., hydrochloride acid, acetic acid, phosphoric acid, lactic acid, tartaric acid, citric acid) or bases (e.g., sodium hydroxide).

In one embodiment, the composition further comprises at least one taste enhancing/masking agent. Taste enhancing/masking agents are organolaeptic additives used for improvement of taste.

In one embodiment, the taste enhancing/masking agents can be a sweetener, for example, sodium saccharin, sucrose, glucose, fructose, aspartame and/or sucralose, in a concentration range of 0.05-0.5% (w/v). In a preferred embodiment, said taste enhancing/masking agent is sucralose, preferably with a concentration of 0.05 percent (w/v).

In one embodiment, the composition further comprises at least one flavouring agent, i.e. a flavour enhancer. The definition of “flavour enhancer” is laid down in point 14 of Annex I of Regulation (EC) No 1333/20082 on food additives: “flavour enhancers are substances which enhance the existing taste and/or odour of a foodstuff”.

In one embodiment, the flavouring agent can be of any fruit such as lemon, apple, banana, pineapple, orange, berries, apricot, cherry, and/or vanilla, peppermint, cinnamon, or any other pharmaceutically acceptable flavouring excipient. In a preferred embodiment, said flavouring agent is vanilla, more preferably in a concentration in the range of 0.05 percent to 0.2 percent (w/v), even more preferably 0.1 percent (w/v).

In a preferred embodiment, the pharmaceutical composition comprises branaplam or a pharmaceutically acceptable salt thereof in a concentration of 3.5 mg/ml, 2-hydroxypropyl-beta-cyclodextrin in a concentration of 17.5 percent (w/v), sucralose in a concentration of 0.05 percent (w/v), vanilla in a concentration of 0.1 percent (w/v), water. Preferably, the pH of the composition is in the range of about 4 to about 7, more preferably about 4.

In a preferred embodiment, the pharmaceutical composition comprises branaplam, or a pharmaceutically acceptable salt thereof (e.g. hydrochloride salt) in a concentration of 1 mg/ml to 30 mg/ml (e.g., 3.5 mg/ml) of branaplam [i.e. compound of formula (I), as herein, in the free form] and a pharmaceutically acceptable cyclodextrin (e.g., 2-hydroxypropyl-beta-cyclodextrin) in a concentration of 17.5 percent (w/v), in water. Preferably, the pH of the composition is in the range of about 4 to about 7, more preferably about 4. Amounts (i.e. mg/ml) referring to an amount of branaplam [i.e. compound of formula (I), as herein, in the free form] will be adapted accordingly if a pharmaceutically acceptable salt thereof (e.g., hydrochloride salt) is used.

In one embodiment, the pharmaceutical composition comprises branaplam, or a pharmaceutically acceptable salt thereof (e.g. hydrochloride salt), in a concentration of 1 mg/ml to 30 mg/ml (e.g., 3.5 mg/ml) of branaplam [i.e. compound of formula (I), as herein, in the free form], a pharmaceutically acceptable cyclodextrin (e.g., 2-hydroxypropyl-beta-cyclodextrin) in a concentration of 17.5 percent (w/v) and at least one taste-masking agent (e.g. sucralose) in a concentration of from 0.05% to 0.5% (w/v), for example 0.05% (w/v), in water. Preferably, the pH of the composition is in the range of about 4 to about 7, more preferably about 4. Amounts (i.e. mg/ml) referring to an amount of branaplam [i.e. compound of formula (I), as herein, in the free form] will be adapted accordingly if a pharmaceutically acceptable salt thereof (e.g., hydrochloride salt) is used.

In one embodiment, the pharmaceutical composition comprises branaplam, or a pharmaceutically acceptable salt thereof (e.g. hydrochloride salt), in a concentration of 1 mg/ml to 30 mg/ml (e.g., 3.5 mg/ml) of branaplam [i.e. compound of formula (I), as herein, in the free form], a pharmaceutically acceptable cyclodextrin (e.g., 2-hydroxypropyl-beta-cyclodextrin) in a concentration of 17.5 percent (w/v), at least one taste-masking agent (e.g. sucralose) in a concentration of from 0.05% to 0.5% (w/v), for example 0.05 (w/v), and at least one flavouring agent, for example vanilla, in a concentration of from 0.05% to 0.2% (w/v), for example 0.1% (w/v), in water. Preferably, the pH of the composition is in the range of about 4 to about 7, more preferably about 4. Amounts (i.e. mg/ml) referring to an amount of branaplam [i.e. compound of formula (I), as herein, in the free form] will be adapted accordingly if a pharmaceutically acceptable salt thereof (e.g., hydrochloride salt) is used.

In a preferred embodiment, the pharmaceutical composition comprises branaplam, or a pharmaceutically acceptable salt thereof (e.g. hydrochloride salt) in a concentration of 1 mg/ml to 30 mg/ml (e.g., 3.5 mg/ml) of branaplam [i.e. compound of formula (I), as herein, in the free form] and a pharmaceutically acceptable cyclodextrin (e.g., 2-hydroxypropyl-beta-cyclodextrin) in a concentration of 10 percent (w/v), in water. Preferably, the pH of the composition is in the range of about 4 to about 7, more preferably about 4. Amounts (i.e. mg/ml) referring to an amount of branaplam [i.e. compound of formula (I), as herein, in the free form] will be adapted accordingly if a pharmaceutically acceptable salt thereof (e.g., hydrochloride salt) is used.

In one embodiment, the pharmaceutical composition comprises branaplam, or a pharmaceutically acceptable salt thereof (e.g. hydrochloride salt), in a concentration of 1 mg/ml to 30 mg/ml (e.g., 3.5 mg/ml) of branaplam [i.e. compound of formula (I), as herein, in the free form], a pharmaceutically acceptable cyclodextrin (e.g., 2-hydroxypropyl-beta-cyclodextrin) in a concentration of 10 percent (w/v) and at least one taste-masking agent (e.g. sucralose) in a concentration of from 0.05% to 0.5% (w/v), for example 0.05% (w/v), in water. Preferably, the pH of the composition is in the range of about 4 to about 7, more preferably about 4. Amounts (i.e. mg/ml) referring to an amount of branaplam [i.e. compound of formula (I), as herein, in the free form] will be adapted accordingly if a pharmaceutically acceptable salt thereof (e.g., hydrochloride salt) is used.

In one embodiment, the pharmaceutical composition comprises branaplam, or a pharmaceutically acceptable salt thereof (e.g. hydrochloride salt), in a concentration of 1 mg/ml to 30 mg/ml (e.g., 3.5 mg/ml) of branaplam [i.e. compound of formula (I), as herein, in the free form], a pharmaceutically acceptable cyclodextrin (e.g., 2-hydroxypropyl-beta-cyclodextrin) in a concentration of 10 percent (w/v), at least one taste-masking agent (e.g. sucralose) in a concentration of from 0.05% to 0.5% (w/v), for example 0.05 (w/v), and at least one flavouring agent, for example vanilla, in a concentration of from 0.05% to 0.2% (w/v), for example 0.1% (w/v), in water. Preferably, the pH of the composition is in the range of about 4 to about 7, more preferably about 4. Amounts (i.e. mg/ml) referring to an amount of branaplam [i.e. compound of formula (I), as herein, in the free form] will be adapted accordingly if a pharmaceutically acceptable salt thereof (e.g., hydrochloride salt) is used.

Interaction of HP-b-CD with preservatives (e.g., Parabens, Chlorobutanol, Benzalkonium chloride) is well known in literature. Loss of antimicrobial activity against microorganisms in the presence of HP-b-CD was observed, thus leading to an increase in minimum inhibitory concentration (MIC) of preservatives. As a result, increasing the levels of preservatives in the formulation is required in order to comply with microbiological tests. HP-b-CD-preserved formulations require optimization of HP-b-CD:Drug ratio and preservatives' selection in order to minimize the interaction of HP-b-CD with the preservative. However, HP-b-CD optimization (i.e., decreasing HP-b-CD levels) increases the risk of drug precipitation during storage and shelf life.

There is a limited number of approved preservatives available for multi-use oral products suitable for the target infant population. In addition, branaplam shows incompatibility with potassium sorbate with antibacterial and antifungal properties, thus demanding suitable alternatives in particular against yeast and moulds.

In one embodiment, the pharmaceutical composition is free or substantially free of preservatives. In this context, the term “substantially” means that preservatives are not detectable in the composition, or only in concentrations which are generally considered irrelevant with regard to any preservation effects. Whether a composition is effectively preserved may be determined according to tests known to those skilled in the art, such as the test for preservative efficacy (USP <51>). In one embodiment, “preservatives” are compounds that inhibit microbial growth and are typically added to dispersions to prevent microbes from growing. In another embodiment, the term “preservatives”, as used herein, refers to compounds that are added, particularly to aqueous preparations, to prevent proliferation or limit microbial contamination which, during normal conditions of storage and use, particularly for multidose containers, could occur in a product and present a hazard to the patient from infection and spoilage of the preparation. Typically, amounts of preservatives needed to pass anti-microbial effectiveness testing as described by USP and EU methodology are used to test appropriate preservative levels. Preservatives include but are not limited to propionic acid, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

As used herein, the term “preservative-free” or “preservative free” or “free of preservative(s)” means that no preservative is intentionally added to (or present in) the formulation or pharmaceutical composition.

In a second aspect, the present invention relates to the pharmaceutical composition of the first aspect for use in treating, preventing or ameliorating a SMN-deficiency-related condition, preferably SMA. In a preferred embodiment, said composition is orally administered.

In a third aspect, the present invention relates to a method for treating, preventing or ameliorating a SMN-deficiency-related condition, preferably SMA, comprising administering to a subject in need thereof an effective amount of the pharmaceutical composition of the first aspect. In one embodiment, said composition is administered via an enteral feeding tube. In another embodiment, said composition is orally administered.

In a fourth aspect, the present invention relates to the pharmaceutical composition of the first aspect for the manufacture of a medicament for the treatment or prevention or amelioration of a SMN-deficiency-related condition.

In one embodiment, a pharmaceutical composition of the present invention is administered at a dose of about 0.625 mg/kg to about 3.125 mg/kg of branaplan in free form or in the form of a pharmaceutically acceptable salt. For example, a pharmaceutical composition of the present invention is administered as single dose of about 0.625 mg/kg, about 1.25 mg/kg, about 2.5 mg/kg or about 3.125 mg/kg of branaplan in free form or in the form of a pharmaceutically acceptable salt. In another embodiment said doses are preferably about 0.625 mg/kg, about 1.25 mg/kg, about 2.5 mg/kg or about 3.125 mg/kg of branaplan in free form. Said doses are administered once a week, twice a week or every other day. Preferably, a pharmaceutical composition of the present invention is administered once a week.

In another embodiment, a pharmaceutical composition of the present invention is administered at a dose of from 0.625 mg/kg (or 12 mg/m²) to 3.125 mg/kg (or 60 mg/m²) of branaplam [i.e. compound of formula (I), as herein, in the free form]. For example, a pharmaceutical composition of the present invention is administered as single dose of 0.625 mg/kg (or 12 mg/m²), 1.25 mg/kg (or 24 mg/m²), 2.5 mg/kg (or 48 mg/m²) or 3.125 mg/kg (or 60 mg/m²) of branaplam [i.e. compound of formula (I), as herein, in the free form]. In another embodiment said doses are preferably 0.625 mg/kg (or 12 mg/m²), 1.25 mg/kg (or 24 mg/m²), 2.5 mg/kg (or 48 mg/m²) or 3.125 mg/kg (or 60 mg/m²) of branaplam [i.e. compound of formula (I), as herein, in the free form]. Said doses are administered once a week, twice a week or every other day. Preferably, a pharmaceutical composition of the present invention is administered once a week. Amounts referring to the amount of branaplam [i.e. compound of formula (I), as herein, in the free form] will be adapted accordingly if a pharmaceutically acceptable salt thereof (e.g., hydrochloride salt) is used. The doses specified per square meter (e.g. mg/m²) are based on the body surface area (BSA) as calculated according to the below formula using the weight and height of the subject.

BSA (m²)=(Weight (kg)^{0.425}×Height (cm)^{0.725})×0.07184

In a fifth aspect, the invention relates to a method of treating, preventing or ameliorating a SMN-deficiency-related condition, preferably SMA comprising administration of branaplan, or a pharmaceutically acceptable salt thereof, at a dose of about 0.625 mg/kg to about 3.125 mg/kg once a week, twice a week or every other day. For example, in a method of the present invention branaplan in free form or in the form of a pharmaceutically acceptable salt is administered as single dose of about 0.625 mg/kg, about 1.25 mg/kg, about 2.5 mg/kg or about 3.125 mg/kg. In a preferred embodiment, said dose is administered once a week. In another embodiment, said dose is preferably 0.625 mg/kg or 2.5 mg/kg. In another embodiment said doses are preferably about 0.625 mg/kg, about 1.25 mg/kg, about 2.5 mg/kg or about 3.125 mg/kg of branaplan in free form. In another embodiment, said dose is administered as a pharmaceutical composition of the present invention. In another embodiment said dose is administered orally or via an enteral feeding tube. In a preferred embodiment, said dose is administered orally.

In another aspect, the invention relates to a method of treating, preventing or ameliorating a SMN-deficiency-related condition, preferably SMA comprising administration of branaplam, or a pharmaceutically acceptable salt thereof, at a dose of from 0.625 mg/kg (or 12 mg/m²) to 3.125 mg/kg (or 60 mg/m²) of branaplam [i.e. compound of formula (I), as herein, in the free form] once a week, twice a week or every other day. For example, in a method of the present invention branaplam is administered as single dose of 0.625 mg/kg (or 12 mg/m²), 1.25 mg/kg (or 24 mg/m²), 2.5 mg/kg (or 48 mg/m²) or 3.125 mg/kg (or 60 mg/m²) of branaplam [i.e. compound of formula (I), as herein, in the free form]. In a preferred embodiment, said dose is administered once a week. In another embodiment, said dose is preferably 0.625 mg/kg (or 12 mg/m²) or 2.5 mg/kg (or 48 mg/m²) of branaplam [i.e. compound of formula (I), as herein, in the free form]. In another embodiment said doses are preferably 0.625 mg/kg (or 12 mg/m²), 1.25 mg/kg (or 24 mg/m²), 2.5 mg/kg (or 48 mg/m²) or 3.125 mg/kg (or 60 mg/m²) of branaplam [i.e. compound of formula (I), as herein, in the free form]. In another embodiment, said dose is administered as a pharmaceutical composition of the present invention. In another embodiment said dose is administered orally or via an enteral feeding tube. In a preferred embodiment, said dose is administered orally. Amounts referring to the amount of branaplam [i.e. compound of formula (I), as herein, in the free form] will be adapted accordingly if a pharmaceutically acceptable salt thereof (e.g., hydrochloride salt) is used. The doses specified per square meter (e.g. mg/m²) are based on the body surface area (BSA) as calculated according to the formula, herein above, using the weight and height of the subject.

In a sixth aspect, the present invention relates to branaplan, or a pharmaceutically acceptable salt thereof, for use in treating, preventing or ameliorating a SMN-deficiency-related condition, preferably SMA, wherein branaplan or a pharmaceutically acceptable salt thereof is administered at a dose of about 0.625 mg/kg to about 3.125 mg/kg once a week, twice a week or every other day. For example, branaplan in free form or in the form of a pharmaceutically acceptable salt is administered as single dose of about 0.625 mg/kg, about 1.25 mg/kg, about 2.5 mg/kg or about 3.125 mg/kg. In a preferred embodiment, said dose is administered once a week. In another embodiment, said dose is preferably 0.625 mg/kg or 2.5 mg/kg of branaplan in free form or in the form of a pharmaceutically acceptable salt thereof. In another embodiment said doses are preferably about 0.625 mg/kg, about 1.25 mg/kg, about 2.5 mg/kg or about 3.125 mg/kg of branaplan in free form. In another embodiment, said dose is administered as a pharmaceutical composition of the present invention. In another embodiment said dose is administered orally or via an enteral feeding tube. In a preferred embodiment, said dose is administered orally.

In another aspect, the present invention relates to branaplam, or a pharmaceutically acceptable salt thereof, for use in treating, preventing or ameliorating a SMN-deficiency-related condition, preferably SMA, wherein branaplam is administered at a dose of 0.625 mg/kg (or 12 mg/m²) to 3.125 mg/kg (or 60 mg/m²) of branaplam [i.e. compound of formula (I), as herein, in the free form] once a week, twice a week or every other day. For example, branaplam is administered as single dose of 0.625 mg/kg (or 12 mg/m²), 1.25 mg/kg (or 24 mg/m²), 2.5 mg/kg (or 48 mg/m²) or 3.125 mg/kg (or 60 mg/m²) of branaplam [i.e. compound of formula (I), as herein, in the free form]. In a preferred embodiment, said dose is administered once a week. In another embodiment, said dose is preferably 0.625 mg/kg (or 12 mg/m²) or 2.5 mg/kg (or 48 mg/m²) of branaplam [i.e. compound of formula (I), as herein, in the free form]. In another embodiment said doses are preferably 0.625 mg/kg (or 12 mg/m²), 1.25 mg/kg (or 24 mg/m²), 2.5 mg/kg (or 48 mg/m²) or 3.125 mg/kg (or 60 mg/m²) of branaplam [i.e. compound of formula (I), as herein, in the free form]. In another embodiment, said dose is administered as a pharmaceutical composition of the present invention. In another embodiment said dose is administered orally or via an enteral feeding tube. In a preferred embodiment, said dose is administered orally. Amounts referring to the amount of branaplam [i.e. compound of formula (I), as herein, in the free form] will be adapted accordingly if a pharmaceutically acceptable salt thereof (e.g., hydrochloride salt) is used. The doses specified per square meter (e.g. mg/m²) are based on the body surface area (BSA) as calculated according to the formula, herein above, using the weight and height of the subject.

In a seventh aspect, the present invention relates to the use of branaplan, or a pharmaceutically acceptable salt thereof, for the manufacture of a medicament for the treatment or prevention or amelioration of a SMN-deficiency-related condition wherein the medicament is administered at a dose of about 0.625 mg/kg to about 3.125 mg/kg once a week, twice a week or every other day. For example, branaplan in free form or in the form of a pharmaceutically acceptable salt is administered as single dose of about 0.625 mg/kg, about 1.25 mg/kg, about 2.5 mg/kg or about 3.125 mg/kg. In a preferred embodiment, said dose is administered once a week. In another embodiment, said dose is preferably 0.625 mg/kg or 2.5 mg/kg of branaplan in free form or in the form of a pharmaceutically acceptable salt thereof. In another embodiment said doses are preferably about 0.625 mg/kg, about 1.25 mg/kg, about 2.5 mg/kg or about 3.125 mg/kg of branaplan in free form. In another embodiment, said dose is administered as a pharmaceutical composition of the present invention. In another embodiment said dose is administered orally or via an enteral feeding tube. In a preferred embodiment, said dose is administered orally.

In another aspect, the present invention relates to the use of branaplam, or a pharmaceutically acceptable salt thereof, for the manufacture of a medicament for the treatment or prevention or amelioration of a SMN-deficiency-related condition, such as spinal muscular atrophy (SMA), wherein the medicament is administered at a dose of 0.625 mg/kg (or 12 mg/m²) to 3.125 mg/kg (or 60 mg/m²) of branaplam [i.e. compound of formula (I), as herein, in the free form] once a week, twice a week or every other day. For example, branaplam is administered as single dose of 0.625 mg/kg (or 12 mg/m²), 1.25 mg/kg (or 24 mg/m²), 2.5 mg/kg (or 48 mg/m²) or 3.125 mg/kg (or 60 mg/m²) of branaplam [i.e. compound of formula (I), as herein, in the free form]. In a preferred embodiment, said dose is administered once a week. In another embodiment, said dose is preferably 0.625 mg/kg (or 12 mg/m²) or 2.5 mg/kg (or 48 mg/m²) of branaplam [i.e. compound of formula (I), as herein, in the free form]. In another embodiment said doses are preferably 0.625 mg/kg (or 12 mg/m²), 1.25 mg/kg (or 24 mg/m²), 2.5 mg/kg (or 48 mg/m²) or 3.125 mg/kg (or 60 mg/m²) of branaplam [i.e. compound of formula (I), as herein, in the free form]. In another embodiment, said dose is administered as a pharmaceutical composition of the present invention. In another embodiment said dose is administered orally or via an enteral feeding tube. In a preferred embodiment, said dose is administered orally. Amounts referring to the amount of branaplam [i.e. compound of formula (I), as herein, in the free form] will be adapted accordingly if a pharmaceutically acceptable salt thereof (e.g., hydrochloride salt) is used. The doses specified per square meter (e.g. mg/m²) are based on the body surface area (BSA) as calculated according to the formula, herein above, using the weight and height of the subject.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

“Solubilizers” include compounds such as Cremophor RH 40, Tween 80, propylene glycol, triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, cyclodextrins, ethanol, n-butanol, isopropyl alcohol, cholesterol, bile salts, polyethylene glycol 200 to 600, glycofurol, transcutol, propylene glycol, and dimethyl isosorbide, miglyol, glycerin, glycerol, and the like. Typically, solubilizers are used in a concentration in the range of about 1 percent to 25 percent (e.g. w/v). In a preferred embodiment, 2-hydroxypropyl-beta-cyclodextrin in a concentration of 17.5 percent (e.g. w/v) is used as solubilizer.

One or more pharmaceutically acceptable pH adjusting agents and/or buffering agents can be included in a composition of the invention, including acids such as acetic, boric, citric, lactic, phosphoric and hydrochloric acids; bases such as sodium hydroxide, sodium borate, sodium citrate, sodium acetate, sodium lactate and trishydroxymethylaminomethane; and buffers such as citrate/dextrose, sodium bicarbonate and ammonium chloride. Such acids, bases and buffers are included in an amount required to maintain pH of the composition.

The invention also provides methods of preparing a liquid formulation. A first method comprises the steps of: forming a first aqueous solution comprising a cyclodextrin and/or a cyclodextrin derivative (e.g. 2-hydroxypropyl-beta-cyclodextrin); forming a suspension comprising active agent (i.e. branaplam or a pharmaceutically acceptable salt thereof); and mixing said solution and suspension to form the liquid formulation. A second method is similar to the first step except that the active agent is added directly to the first solution without formation of the suspension. A third method is similar to the first except that the cyclodextrin and/or cyclodextrin derivative is added directly to the suspension without formation of the first solution. A fourth method comprises the steps of: adding a suspension comprising active agent to a powdered or particulate cyclodextrin and/or cyclodextrin derivative. A fifth method comprises the steps of: adding the active agent directly to the powdered or particulate cyclodextrin and/or cyclodextrin derivative; and adding a second solution. A sixth method comprises the steps of: creating the liquid formulation by any of the above methods and then isolating a solid material by lyophilisation, spray-drying, spray-freeze-drying, antisolvent precipitation, a process utilizing a supercritical or near supercritical fluid, or other methods known to those of ordinary skill in the art to make a powder for reconstitution.

A liquid formulation of the invention may also be converted to a solid formulation for reconstitution. A reconstitutable solid pharmaceutical composition according to the invention comprises an active agent, a derivatized cyclodextrin and optionally at least one other pharmaceutical excipient. This composition is reconstituted with an aqueous liquid to form a liquid formulation that is preserved. The composition can comprise an admixture of a solid derivatized cyclodextrin and an active agent-containing solid and optionally at least one solid pharmaceutical excipient, such that a major portion of the active agent is not complexed with the derivatized cyclodextrin prior to reconstitution. Alternatively, the composition can comprise a solid mixture of a derivatized cyclodextrin and an active agent, wherein a major portion of the active agent is complexed with the derivatized cyclodextrin prior to reconstitution.

The reconstitutable formulation can be prepared according to any of the following processes. A liquid formulation of the invention is first prepared, then a solid is formed by lyophilization (freeze-drying), spray-drying, spray freeze-drying, antisolvent precipitation, various processes utilizing supercritical or near supercritical fluids, or other methods known to those of ordinary skill in the art to make a solid for reconstitution.

Although not necessary, the formulation of the present invention may include a conventional preservative, antioxidant, buffering agent, acidifying agent, alkalizing agent, colorant, solubility-enhancing agent, complexation enhancing agent, electrolyte, glucose, stabilizer, tonicity modifier, bulking agent, antifoaming agent, oil, emulsifying agent, cryoprotectant, plasticizer, flavours, sweeteners, other excipients known by those of ordinary skill in the art for use in preserved formulations, or a combination thereof.

As used herein, a conventional preservative is a compound used to at least reduce the rate at which bioburden increases, but preferably maintains bioburden steady or reduces bioburden after contamination. Such compounds include, by way of example and without limitation, benzalkonium chloride, benzethonium chloride, benzoic acid, benzyl alcohol, cetylpyridinium chloride, chlorobutanol, phenol, phenylethyl alcohol, phenylmercuric nitrate, phenylmercuric acetate, thimerosal, metacresol, myristylgamma picolinium chloride, potassium benzoate, sodium benzoate, sodium propionate, sorbic acid, thymol, and methyl, ethyl, propyl or butyl parabens and others known to those of ordinary skill in the art.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings) and/or all of the steps of any method or process so disclosed may be combined in any combination, except combinations where at least some of such features and/or steps are mutually exclusive. Any embodiments specifically and explicitly recited herein may form the basis of a disclaimer either alone or in combination with one or more further embodiments. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The following examples are intended to illustrate the invention and are not to be construed as being limitations thereon. Temperatures are given in degrees Celsius. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4^{th} Ed. 1952, Methods of Organic Synthesis, Thieme, Volume 21). In particular, branaplam can be produced by organic synthesis methods disclosed in WO 2014/028459, under example 17-13, which is hereby incorporated by reference.

### Further Embodiments

Embodiment 1: A pharmaceutical composition comprising
A) The compound of formula (I)

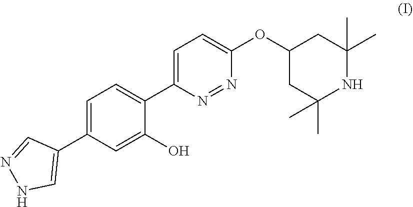 [SMILES: CC1(C)CC(Oc2ccc(-c3ccc(-c4cn[nH]c4)cc3O)nn2)CC(C)(C)N1]

or a pharmaceutically acceptable salt thereof, and
B) a pharmaceutically acceptable cyclodextrin or combination of pharmaceutically acceptable cyclodextrins.

Embodiment 2: The pharmaceutical composition of embodiment 1, wherein the compound of formula (I) is in its hydrochloride salt form.

Embodiment 3: The pharmaceutical composition of embodiment 1 or 2, wherein the cyclodextrin is a beta-cyclodextrin.

Embodiment 4: The pharmaceutical composition according to any one of the embodiments 1 to 3, wherein the pharmaceutically acceptable cyclodextrin (B) is selected from the group consisting of 2-hydroxypropyl-beta-cyclodextrin, sulfobutylether-beta-cyclodextrin, beta-cyclodextrin, methyl-beta-cyclodextrin, hydroxyethyl-beta-cyclodextrin, ethyl-beta-cyclodextrin, butyl-beta-cyclodextrin, Succinyl-(2-hydroxypropyl)-beta-cyclodextrin, heptakis(2,3,6-tri-O-methyl)-beta-cyclodextrin, heptakis(2,3,6-tri-O-benzoyl)-beta-cyclodextrin, beta-cyclodextrin phosphate sodium salt, beta-cyclodextrin sulphate sodium salt, triacetyl-beta-cyclodextrin, heptakis(6-O-sulfo)-beta-cyclodextrin heptasodium salt, carboxymethyl-beta-cyclodextrin sodium salt, sulfobutylether-beta-cyclodextrin sodium salt, and 6-O-p-toluenesulfonyl-beta-cyclodextrin.

Embodiment 5: The pharmaceutical composition according to any one of preceding embodiments, wherein the composition is a liquid composition, for example an aqueous liquid composition.

Embodiment 6: The pharmaceutical composition according to any one of preceding embodiments, wherein cyclodextrin (B) is 2-hydroxypropyl-beta-cyclodextrin.

Embodiment 7: The pharmaceutical composition according to any one of embodiments 1-5, wherein cyclodextrin (B) is sulfobutylether-beta-cyclodextrin.

Embodiment 8: The pharmaceutical composition according to any one of preceding embodiments, wherein the concentration of the compound of formula I or any pharmaceutically acceptable salt thereof is in the range of about 1 mg/ml to about 30 mg/ml.

Embodiment 9: The pharmaceutical composition according to embodiment 8, wherein the concentration of the compound of formula I or any pharmaceutically acceptable salt thereof is in the range of about 3 mg/ml to about 10 mg/ml.

Embodiment 10: The pharmaceutical composition according to any one of preceding embodiments, wherein the cyclodextrin is present in a concentration in the range of 0.1 percent to 70 percent (w/v).

Embodiment 11: The pharmaceutical composition according to embodiment 10, wherein the cyclodextrin is present in a concentration in the range of 2 percent to 25 percent (w/v).

Embodiment 12: The pharmaceutical composition according to any one of preceding embodiments, wherein the pH of the composition is in the range of 3.5-9.

Embodiment 13: The pharmaceutical composition according to embodiment 12, wherein the pH of the composition is about 4.

Embodiment 14: The pharmaceutical composition according to any one of preceding embodiments, said composition comprising:
A) the compound of formula I or a pharmaceutically acceptable salt thereof in a concentration of 1 mg/ml to 30 mg/ml,
B) 2-hydroxypropyl-beta-cyclodextrin in a concentration in the range of 2 percent to 25 percent (w/v), and wherein the pH of the composition is about 4.0.

Embodiment 15: The pharmaceutical composition according to any one of preceding embodiments, wherein the composition further comprises at least one taste-masking agent.

Embodiment 16: The pharmaceutical composition according to embodiment 15, wherein the taste-masking agent is sucralose.

Embodiment 17: The pharmaceutical composition according to any one of preceding embodiments, wherein the composition further comprises at least one flavouring agent.

Embodiment 18: The pharmaceutical composition according to embodiment 17, wherein the flavouring agent is vanilla.

Embodiment 19: The pharmaceutical composition according to any one of preceding embodiments, said composition comprising:
a) the hydrochloride salt of the compound of formula (I) in a concentration of 3.5 mg/ml,
b) 2-hydroxypropyl-beta-cyclodextrin in a concentration of 17.5 percent (w/v),
c) sucralose in a concentration of 0.05 percent (w/v),
d) vanilla in a concentration of 0.1 percent (w/v)
e) water

and wherein the pH of the composition is about 4.0 or higher.

Embodiment 20: The pharmaceutical composition according to any one of the preceding embodiments, wherein the composition is substantially free of preservatives.

Embodiment 21: The pharmaceutical composition according to any one of the preceding embodiments for use as a medicament.

Embodiment 22: The pharmaceutical composition according to any one of the preceding embodiments, wherein said composition is to be administered orally.

Embodiment 23: The pharmaceutical composition according to any one of the preceding embodiments for use in treatment or prevention or amelioration of a SMN-deficiency-related condition.

Embodiment 24: The pharmaceutical composition for use according to embodiment 23, wherein said SMN-deficiency-related condition is Spinal Muscular Atrophy (SMA).

Embodiment 25: A method to treat, prevent or ameliorate a SMN-deficiency-related condition, comprising administering to a subject in need thereof an effective amount of a composition according to any one of the preceding embodiments.

Embodiment 26: The method of embodiment 25, wherein said SMN-deficiency-related condition is Spinal Muscular Atrophy (SMA).

Embodiment 27: The method of embodiment 25 wherein the composition is administered at a dose of about 0.625 mg/kg to about 3.125 mg/kg of branaplan, in free form or in the form of a pharmaceutically acceptable salt, per weight of subject.

Embodiment 28: The use of a pharmaceutical composition according to any one of embodiments 1-20, or 22, for the manufacture of a medicament for the treatment or prevention or amelioration of a SMN-deficiency-related condition.

### Abbreviations

HP-b-CD=2-hydroxypropyl-beta-cyclodextrin

DS=(cyclodextrin's) average degree of substitution

Captisol=sulfobutylether β-cyclodextrin sodium salt

w/v=weight per volume. Where the concentration is expressed as a percentage, N % w/v means there is N grams of the solute in 100 mililiters of the entire solution.

SD (as used in Tables 2a, 3a, 3 and 4)=standard deviation.

q.s.=quantum sufficit, i.e. “as much as needed”.

mg/mL=milligram/milliliter.

mL=ml=milliliter

RT=room temperature (25±3° C.)

AET=Antimicrobiologic Effectiveness Testing

n.a. (as used in Table 6a and Table 7)=not applicable.

### EXAMPLES

### Examples 1-8

Examples 1-8 describe some of the preferred embodiments of the present invention. The details of oral formulations of branaplam as in said examples are given in Tables 1-4.

TABLE 1
Oral formulation of branaplam according to Example 1.

| Ingredients | Amount |
|---|---|
| Branaplam monohydrochloride salt | 1-30 mg/ml |
| 2-hydroxypropyl-beta-cyclodextrin | 0.1-70 percent (w/w) |
| Hydrochloride/acetic/phosphoric/lactic/ | q.s. to pH 3.5-9 |
| tartaric/citric acid | |
| Sodium hydroxide | |
| Water | q.s. |
| pH adjusted to | 3.5-9 |

TABLE 2
Phase solubility data of branaplam in 2-hydroxypropyl-
beta-cyclodextrins solutions (degree of substitution 6.1)

| | Hydrochloride/acetic/ | | | Branaplam base solubility (mg/ml) | | |
|---|---|---|---|---|---|---|
| | phosphoric/lactic/ | Sodium | HP-b-CD | HP-b-CD DS 6.1 | | |
| Example | tartaric/citric acid | hydroxide | (% w/w) | pH 3.5 | pH 5.0 | pH 6.0 |

| | | | | | |
|---|---|---|---|---|---|
| 2 | q.s. to adjust the pH | 0.0 | 1.03 | 0.91 | 0.81 |
| 3 | q.s. to adjust the pH | 3.0 | 3.17 | 2.87 | 3.09 |
| 4 | q.s. to adjust the pH | 8.0 | 5.84 | 5.70 | 5.57 |
| 5 | q.s. to adjust the pH | 12.0 | 7.67 | 7.57 | 7.98 |
| 6 | q.s. to adjust the pH | 17.5 | 9.27 | 10.45 | 10.61 |

TABLE 3
Oral formulation of branaplam comprising up to 25.0%
(w/w) 2-hydroxypropyl-beta-cyclodextrin with a
degree of substitution of 4.6 at a pH of 4.

| | Hydrochloride/ | | | | |
|---|---|---|---|---|---|
| | acetic/ | | | Branaplam base | |
| | phosphoric/ | | | solubility (mg/mL) | |
| | lactic/tartaric/ | Sodium | HP-b-CD | DS 4.6 | |
| Example | citric acid | hydroxide | (% w/w) | Average | SD |

| | | | | |
|---|---|---|---|---|
| 2 | q.s. to adjust the pH = 4.0 | 0.0 | 0.75 | 0.02 |
| 3 | q.s. to adjust the pH = 4.0 | 3.0 | 2.77 | 0.02 |
| 4 | q.s. to adjust the pH = 4.0 | 8.0 | 5.32 | 0.07 |
| 5 | q.s. to adjust the pH = 4.0 | 12.0 | 7.09 | 0.18 |
| 6 | q.s. to adjust the pH = 4.0 | 17.5 | 9.51 | 0.11 |
| 7 | q.s. to adjust the pH = 4.0 | 25.0 | 12.56 | 0.17 |

TABLE 4
Oral formulation of branaplam comprising up to 25.0%
(w/w) 2-hydroxypropyl-beta-cyclodextrin with a
degree of substitution of 6.3 at a pH of 4.

| | Hydrochloride/ | | | | |
|---|---|---|---|---|---|
| | acetic/ | | | Branaplam base | |
| | phosphoric/ | | | solubility (mg/mL) | |
| | lactic/tartaric/ | Sodium | HP-b-CD | DS 6.3 | |
| Example | citric acid | hydroxide | (% w/w) | Average | SD |

| | | | | |
|---|---|---|---|---|
| 2 | q.s. to adjust the pH = 4.0 | 0.0 | 0.75 | 0.02 |
| 3 | q.s. to adjust the pH = 4.0 | 3.0 | 2.69 | 0.01 |
| 4 | q.s. to adjust the pH = 4.0 | 8.0 | 5.32 | 0.09 |
| 5 | q.s. to adjust the pH = 4.0 | 12.0 | 6.89 | 0.10 |
| 6 | q.s. to adjust the pH = 4.0 | 17.5 | 8.54 | 0.08 |
| 7 | q.s. to adjust the pH = 4.0 | 25.0 | 11.80 | 0.11 |

### Example 8

TABLE 5
Oral formulation of branaplam according to Example 8.

| Ingredients | Amount |
|---|---|
| Branaplam monohydrochloride salt | 3.826 mg/ml |
| 2-hydroxypropyl-beta-cyclodextrin | 17.5 percent (w/w) |
| Hydrochloride acid | q.s. to pH 4 |
| Sodium hydroxide | |
| Water | q.s. |
| pH adjusted to | 4 |

Procedure:

The required amount of 2-hydroxypropyl-beta-cyclodextrin was dissolved in 80% volume of target water and stirred for 30 minutes. The required amount of branaplam was then added to said solution, under stirring, at room temperature. The solution was stirred for 45 minutes after the addition was completed or for longer until a particle-free solution was obtained. Initial pH adjustment was performed using NaOH 0.1M or HCl 0.1M to reach the intended pH (±0.25). The required volume of water was added to the solution to reach the final intended volume and stirred for at least 10 minutes at 25±3° C. after the addition was completed. Final pH adjustment was performed using NaOH 0.1 M or HCL 0.1 M to reach the intended pH.

### Comparative Example 1

Solubility of branaplam was evaluated in excipients, other than cyclodextrin, such as Cremophor RH40, Tween 80, PG, PEG300, and glycerol. These excipients and concentrations were selected as they are suitable for paediatric formulations. None of the tested excipients was able to support development of a formulation comprising branaplam at a concentration high enough (i.e. about 2 mg/ml or higher) to keep the dose volume in a suitable range (Table 6).

TABLE 6
Solubility in selected excipients.

| | Concentration | Solubility (mg/mL) | Solubility (mg/mL) |
|---|---|---|---|
| Excipients | (w/w) | RT | 2-8° C. |
| Cremophor RH | 1.0% | 0.28 | 0.17 |
| 40 | 3.5% | 0.53 | 0.44 |
| | 7.0% | 0.84 | 0.71 |
| | 15.0% | 1.32 | 1.19 |
| Tween 80 | 0.5% | 0.25 | 0.16 |
| | 0.8% | 0.28 | 0.22 |
| | 1.5% | 0.41 | 0.29 |
| | 5.0% | 0.89 | 0.73 |
| Propylene | 5.0% | 0.27 | 0.16 |
| glycol | 10.0% | 0.37 | 0.18 |
| | 25.0% | 0.94 | 0.42 |
| PEG 300 | 5.0% | 0.39 | 0.27 |
| | 10.0% | 0.64 | 0.34 |
| | 25.0% | 1.61 | 0.87 |
| PEG 400 | 5.0% | 0.39 | 0.21 |
| | 10.0% | 0.63 | 0.45 |
| | 25.0% | 1.47 | 0.93 |
| Glycerol | 5.0% | 0.18 | 0.20 |
| | 10.0% | 0.22 | 0.19 |
| | 25.0% | 0.38 | 0.37 |

Based on these results, a formulation based on conventional excipients could not be identified.

### Comparative Example 2

The following excipients were evaluated for their use as preservatives in oral solutions: propionic acid; bronopol; phenol; chlorobutol; benzalkonium chloride; thiomerosal; benzyl alcohol; and parabens. Oral Permissible Daily Exposure (PDE) values of 9.3-22 mg/day for propionic acid, 0.19-0.46 mg/day for bronopol, 0.036-0.084 mg/day for chlorbutol, 0.038-0.091 mg/day for phenol, 2.8 mg/kg/day for methylparaben and 2.0 mg/kg/day for Propylparaben were calculated for paediatric patient populations (new-borns, infants and toddlers). From a toxicological perspective, propionic acid, benzoic acid and parabens were investigated as preservatives for branaplam oral solution. The HP-b-CD concentration was reduced to 7.5% (w/w) and 10% (w/w) in order to minimize the concentration of free HP-b-CD available to interact with the preservatives. Lower concentrations of HP-b-CD showed precipitation after storage in the fridge and therefore are not recommended. The branaplam solution was prepared as described for Examples 1-8, followed by adding the intended preservatives with the specified concentrations.

Table 7 shows the Antimicrobiologic Effectiveness Testing (AET) results of the tested formulations. Multidose formulations must comply with the AET testing. Precipitation was observed when the HP-b-CD concentration was reduced to 7.5% (w/w) and in the presence of benzoic acid. Formulations with 0.2% (w/v) propyl paraben and 0.3% (w/w) methyl paraben and 7.5% (w/w) HP-b-CD failed AET testing. From the tested formulations, only the formulations with propionic acid with or without parabens met the AET specification. However, propionic acid is volatile and with an aversive smell; consequently, its use for paediatric oral solution is not recommended.

TABLE 7
Branaplam multiple dose formulation development: AET results.
LMI070-ORA-Composition [% w/v]

| Formulation | F1 | F2 | F3 | F4 | F5 | F6 | F7 | F8 | F9 | F10 | F11 |
|---|---|---|---|---|---|---|---|---|---|---|---|
| LMI070-AA^{1) 2)} | 0.383 | 0.383 | 0.383 | 0.383 | 0.383% | 0.383% | 0.383% | 0.383% | 0.383% | 0.383% | 0.383% |
| HP-b- | 7.5 | 7.5 | 7.5 | 7.5 | 10.0 | 10.0 | 7.5 | 7.5 | 7.5 | 7.5 | 7.5 |
| Cyclodextrin | | | | | | | | | | | |
| Benzoic acid | 0.05 | 0.1 | 0.2 | | 0.1 | 0.2 | 0.5 | | 0.5 | | |
| Propionic acid | | | | 0.2 | | | | | | 0.5 | 0.5 |
| Metyl paraben | | | | | | | | 0.3 | 0.3 | | 0.3 |
| Propyl paraben | | | | | | | | 0.2 | 0.2 | | 0.2 |
| Final pH | | | | | pH 4.0 ± 0.2 | | | | | | |
| Holding time | <3 | <3 | <3 | <3 | <3 | <3 | n.a. | n.a. | n.a. | n.a. | n.a. |
| [days] | | | | | | | | | | | |
| AET [Pass/fail] | n.a. ³⁾ | n.a. ³⁾ | n.a. ³⁾ | n.a. ³⁾ | n.a. ³⁾ | n.a. ³⁾ | n.a. ⁴⁾ | Fail | n.a. ⁴⁾ | Pass | Pass |

¹⁾LMI070 weight adjustments were performed for drug substance content ≤99.5% and less amount of water for DS compensation.
²⁾0.383% LMI70-AAA corresponds to 3.5 mg LMI070 free base (salt/base ratio 1.093)
³⁾ AET Not performed as holding time was <3 days
⁴⁾ AET Not performed as precipitation was observed, when stored at 2-8° C.

Based on these results, a formulation to support multiple dosing could not be identified.

### Example 9

Taste assessment of branaplam oral solutions with and without sweeteners and flavours was performed in human volunteers. Table 8 shows the level of participants' reported perception of an aversive aftertaste and willingness to take the sample as a medicine for chronic use and the Visual Analogue Scale (VAS) using the scale 0 “Pleasant” and 100 “aversive”. The formulation without any taste-masking or flavouring excipients rated near the midpoint on the continuous VAS scale. The taste of the drug was described as “bitter” and “aversive”, with a particular problem with aftertaste. Addition of 0.05% sucralose and 0.1% vanilla was most effective at taste-masking, and most favoured by the participants with 11 out 12 participants willing to take the formulation in comparison with only 5 willing to take the formulation without any taste-masking or flavouring excipients. The formulation containing 0.05% sucralose and 0.1% vanilla was rated as significantly less aversive (VAS=12.5) compared to the formulation without any taste-masking or flavouring excipients (VAS=54), and no participants rated it negatively on the 5-point categorical facial scale (Table 9). All other formulation combinations that were tested were not as effective at masking the aversive taste and aftertaste of the drug.

TABLE 8
Branaplam aversive aftertaste.

| | | | | | | VAS |
|---|---|---|---|---|---|---|
| | Aversive aftertaste | | | | | overall ratings |
| | Yes, a | Yes - a | | Willing | | (0 “pleasant”, |
| | strong | slight | | to take | | 100 |
| Formulations | aftertaste | aftertaste | No | Yes | No | “aversive”) |
| 17.5% Cyclodextrin, pH | 4 | 6 | 2 | 5 | 7 | 54 |
| 4.0 | | | | | | |
| 17.5% Cyclodextrin, 0.05% | 1 | 9 | 2 | 10 | 2 | 29.5 |
| Sucralose pH 4.0 | | | | | | |
| 17.5% Cyclodextrin, 0.05% | 2 | 7 | 3 | 11 | 1 | 12.5 |
| Sucralose, 0.1% Vanilla | | | | | | |
| pH 4.0 | | | | | | |
| 17.5% Cyclodextrin, 0.05% | 2 | 8 | 2 | 9 | 3 | 19.5 |
| Sucralose, 0.05% sodium | | | | | | |
| sacharine pH 4.0 | | | | | | |
| 17.5% Cyclodextrin, 0.05% | 4 | 7 | 1 | 6 | 6 | 35 |
| Sucralose, 0.05% sodium | | | | | | |
| sacharine, 0.1% Vanilla pH | | | | | | |
| 4.0 | | | | | | |

TABLE 9
Categorical Scale Raw Data.

| | 1 | 2 | 3 | 4 | 5 |
|---|---|---|---|---|---|
| Formulation Samples | [symbol] | [symbol] | [symbol] | [symbol] | [symbol] |
| 17.5% Cyclodextrin, pH 4.0 | 0 | 2 | 3 | 7 | 0 |
| 17.5% Cyclodextrin, 0.05% | 3 | 5 | 3 | 1 | 0 |
| Sucralose pH 4.0 | | | | | |
| 17.5% Cyclodextrin, 0.05% | 4 | 6 | 2 | 0 | 0 |
| Sucralose, 0.1% Vanilla pH | | | | | |
| 4.0 | | | | | |
| 17.5% Cyclodextrin, 0.05% | 4 | 4 | 3 | 1 | 0 |
| Sucralose, 0.05% sodium | | | | | |
| sacharine pH 4.0 | | | | | |
| 17.5% Cyclodextrin, 0.05% | 2 | 4 | 2 | 4 | 0 |
| Sucralose, 0.05% sodium | | | | | |
| sacharine, 0.1% Vanilla pH | | | | | |
| 4.0 | | | | | |

The procedure for preparing the above-mentioned solutions was as follows. The required amount of 2-hydroxypropyl-beta-cyclodextrin was dissolved in 80% volume of target water and stirred for 30 minutes. The required amount of branaplam was then added to said solution, under stirring, at room temperature. The solution was stirred for 45 minutes after the addition was completed or for longer until a particle-free solution was obtained. Initial pH adjustment was performed using NaOH 0.1 M or HCL 0.1 M to reach the intended pH (±0.25). The required amount of sucralose was added to the solution under stirring, at room temperature, and stirring was continued for at least 10 minutes after the addition was completed. The required amount of vanilla was added to the solution under stirring, at room temperature, and stirring was continued for at least 10 minutes after the addition was completed. The required volume of water was added to solution to reach the final intended volume and stirred for at least 10 minutes after the addition was completed. Final pH adjustment was performed using NaOH 0.1 M or HCL 0.1 M to reach the intended pH.

### Examples 10-15

Examples 10-15 describe some of the preferred embodiments of the present invention. The details of oral formulations of branaplam as in said examples are given in Tables 10-12.

TABLE 10
Oral formulation of branaplam according to Example 10.

| Ingredients | Amount | |
|---|---|---|
| Branaplam monohydrochloride salt | 1-40 | mg/ml |
| Captisol ® | 0.1-70 | percent (w/w) |

| | |
|---|---|
| Hydrochloride/acetic/phosphoric/ | q.s. to pH 3.5-9 |
| lactic/tartaric/citric acid | |
| Sodium hydroxide | |
| Water | q.s. |
| pH adjusted to | 3.5-9 |

TABLE 11
Oral formulation of branaplam comprising
up to 17.5% (w/w) Captisol ®.

| | Examples | | | |
|---|---|---|---|---|
| | 11 | 12 | 13 | 14 |
| Ingredients | Amount | | | |
| Captisol ® (% w/w) | 2.5 | 7.5 | 12.5 | 17.5 |
| Branaplam monohydrochloride salt (mg/ml) | 4.5 | 9.3 | 12.7 | 15.0 |

| | |
|---|---|
| Hydrochloride/acetic/phosphoric/lactic/ | q.s. to pH 4 |
| tartaric/citric acid | |
| Sodium hydroxide | |
| Water | q.s. |
| pH adjusted to | 4.0 |

TABLE 12
Oral formulation of branaplam according to Example 15.

| Ingredients | Amount | |
|---|---|---|
| Branaplam monohydrochloride salt | 3.826 | mg/ml |
| Captisol ® | 2.5 | percent (w/w) |

| | |
|---|---|
| Hydrochloride acid | q.s. to pH 4 |
| Sodium hydroxide | |
| Water | q.s. |
| pH adjusted to | 4 |

The required amount of Captisol® was dissolved in 80% volume of target water and stirred for 30 minutes. The required amount of branaplam was then added to said solution, under stirring, at room temperature. The solution was stirred for 45 minutes after the addition was completed or for longer until a particle-free solution (to naked eye) was obtained. Initial pH adjustment was performed using NaOH 0.1 M or HCL 0.1 M to reach the intended pH (±0.25). The required volume of water was added to solution to reach the final intended volume and stirred for at least 10 minutes after the addition was completed. Final pH adjustment was performed using NaOH 0.1 M or HCL 0.1 M to reach the intended pH.

### Example 16

This example provides and exemplary method for the preparation of preservative-free formulations of branaplam.

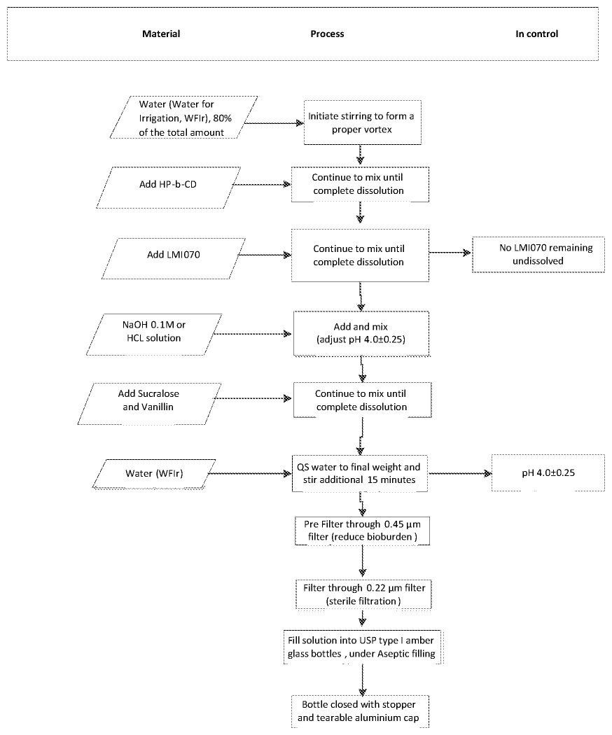

Different branaplam solutions were prepared according to the procedure as described for examples 1-15, each one in a total volume of 40 litres. Each solution was then filtered (pre-filtering) through a 0.45 μm filter. The first 20 mL of the bulk solution through the filter was discarded to confirm the flushing volume for the filter cartridge. The solution was then filtered (sterile filtering) through a 0.22 μm filter. The first 500 mL of the bulk solution through the filter was discarded to confirm the flushing volume for the filter cartridge. The filtered solution was then filled into amber glass vials (6 ml per vial) and closed with lyophilizer stopper and tearable aluminium cap. Optionally, the vials have child-resistant/temper evident closure system. The process for preparation of a branaplam formulation using HP-b-CD is illustrated in FIG. 1. The same process applies when Captisol® is used instead of HP-b-CD.

TABLE 13
Oral formulation of branaplam with reduced cyclodextrin

| Ingredients | Amount | |
|---|---|---|
| Branaplam monohydrochloride salt | 3.826 | mg/ml |
| 2-hydroxypropyl-beta-cyclodextrin | 10.0 | percent (w/w) |

| | |
|---|---|
| Hydrochloride acid | q.s. to pH 4 |
| Sodium hydroxide | |
| Water | q.s. |
| pH adjusted to | 4 |

### Examples 1a-18a

Examples 1a-8a describe some of the preferred embodiments of the present invention. The details of oral formulations of branaplam as in said examples are given in Tables 1a-4a.

TABLE 1a
Phase solubility data of branaplam in 2-hydroxypropyl-beta-cyclodextrins solutions

| | Hydrochloride/acetic/ | | | Branaplam solubility (mg/ml) | | |
|---|---|---|---|---|---|---|
| | phosphoric/lactic/ | Sodium | HP-b-CD | HP-b-CD DS 6.1 | | |
| Example | tartaric/citric acid | hydroxide | (% w/v) | pH 3.5 | pH 5.0 | pH 6.0 |

| | | | | | |
|---|---|---|---|---|---|
| 1a | q.s. to adjust the pH | 0.0 | 1.03 | 0.91 | 0.81 |
| 2a | q.s. to adjust the pH | 3.0 | 3.17 | 2.87 | 3.09 |
| 3a | q.s. to adjust the pH | 8.0 | 5.84 | 5.70 | 5.57 |
| 4a | q.s. to adjust the pH | 12.0 | 7.67 | 7.57 | 7.98 |
| 5a | q.s. to adjust the pH | 17.5 | 9.27 | 10.45 | 10.61 |

TABLE 2a
Oral formulation of branaplam comprising up to
25.0% (w/v) 2-hydroxypropyl-beta-cyclodextrin with
average degree of substitution (DS) of 4.6 at a pH of 4.

| | Hydrochloride/ | | | | |
|---|---|---|---|---|---|
| | acetic/ | | | Branaplam | |
| | phosphoric/ | | | solubility (mg/mL) | |
| | lactic/tartaric/ | Sodium | HP-b-CD | DS 4.6 | |
| Example | citric acid | hydroxide | (% w/v) | Average | SD |

| | | | | |
|---|---|---|---|---|
| 6a | q.s. to adjust the pH = 4.0 | 0.0 | 0.75 | 0.02 |
| 7a | q.s. to adjust the pH = 4.0 | 3.0 | 2.77 | 0.02 |
| 8a | q.s. to adjust the pH = 4.0 | 8.0 | 5.32 | 0.07 |
| 9a | q.s. to adjust the pH = 4.0 | 12.0 | 7.09 | 0.18 |
| 10a | q.s. to adjust the pH = 4.0 | 17.5 | 9.51 | 0.11 |
| 11a | q.s. to adjust the pH = 4.0 | 25.0 | 12.56 | 0.17 |

TABLE 3a
Oral formulation of branaplam comprising up to
25.0% (w/v) 2-hydroxypropyl-beta-cyclodextrin with
average degree of substitution of 6.3 at a pH of 4.

| | Hydrochloride/ | | | | |
|---|---|---|---|---|---|
| | acetic/ | | | Branaplam | |
| | phosphoric/ | | | solubility (mg/mL) | |
| | lactic/tartaric/ | Sodium | HP-b-CD | DS 6.3 | |
| Example | citric acid | hydroxide | (% w/v) | Average | SD |

| | | | | |
|---|---|---|---|---|
| 12a | q.s. to adjust the pH = 4.0 | 0.0 | 0.75 | 0.02 |
| 13a | q.s. to adjust the pH = 4.0 | 3.0 | 2.69 | 0.01 |
| 14a | q.s. to adjust the pH = 4.0 | 8.0 | 5.23 | 0.09 |
| 15a | q.s. to adjust the pH = 4.0 | 12.0 | 6.89 | 0.10 |
| 16a | q.s. to adjust the pH = 4.0 | 17.5 | 8.54 | 0.08 |
| 17a | q.s. to adjust the pH = 4.0 | 25.0 | 11.80 | 0.11 |

### Example 18a

TABLE 4a
Oral formulation of branaplam according to Example 18a.

| Ingredients | Amount | |
|---|---|---|
| Branaplam monohydrochloride salt | 3.826 | mg/ml {*} |
| 2-hydroxypropyl-beta-cyclodextrin | 17.5 | percent (w/v) |

| | |
|---|---|
| Hydrochloride acid | q.s. to pH 4 |
| Sodium hydroxide | |
| Water | q.s. |
| pH adjusted to | 4 |

{*} 3.826 mg of branaplam monohydrochlorde salt corresponds to 3.5 mg of branaplam
(Salt/free form ratio on anhydrous basis 1.093)

Procedure:

The required amount of 2-hydroxypropyl-beta-cyclodextrin was dissolved in 80% volume of target water (i.e. final intended volume) and stirred for 30 minutes. The required amount of branaplam monohydrochloride salt was then added to said solution, under stirring, at room temperature. The solution was stirred for 45 minutes after the addition was completed or for longer until a particle-free solution (to naked eye) was obtained. Initial pH adjustment was performed using NaOH 0.1M or HCl 0.1M to reach the intended pH (±0.25). The required volume of water was added to the solution to reach the final intended volume and stirred for at least 10 minutes at 25±3° C. after the addition was completed. Final pH adjustment was performed using NaOH 0.1 M or HCL 0.1 M to reach the intended pH.

### Comparative Example 1a

Solubility of branaplam was evaluated in excipients, other than cyclodextrin, such as Cremophor RH40, Tween 80, PG, PEG300, and glycerol. These excipients and concentrations were selected as they are suitable for paediatric formulations. None of the tested excipients was able to support development of a formulation comprising branaplam at a concentration high enough (i.e. about 2 mg/ml or higher) to keep the dose volume in a suitable range (Table 5a).

Solubility of branaplam was measured as follows: stock solutions for the excipients at target concentrations were prepared using milli-Q water and adjusted using pH 4.0 buffer. Excess amount of drug substance (i.e. branaplam) was added to individual excipient stock solutions and kept on orbital shaker at set temperature 25° C.±0.5° C. Suspensions were stirred for 24 hours (using a magnetic stirrer) ensuring sufficient swirling and monitoring the temperature. The suspensions were filtered through 0.45 μm nominal pore size filter (e.g. using a PES syringe), and the concentration of branaplam in the filtrate was measured using HPLC (high performance liquid chromatography). The measurements were performed in duplicate and average of the values are reported.

TABLE 5a
Solubility in selected excipients.

| | Concentration | Solubility (mg/mL) | Solubility (mg/mL) |
|---|---|---|---|
| Excipients | (w/v) | RT | 2-8° C. |
| Cremophor RH | 1.0% | 0.28 | 0.17 |
| 40 | 3.5% | 0.53 | 0.44 |
| | 7.0% | 0.84 | 0.71 |
| | 15.0% | 1.32 | 1.19 |
| Tween 80 | 0.5% | 0.25 | 0.16 |
| | 0.8% | 0.28 | 0.22 |
| | 1.5% | 0.41 | 0.29 |
| | 5.0% | 0.89 | 0.73 |
| Propylene | 5.0% | 0.27 | 0.16 |
| glycol | 10.0% | 0.37 | 0.18 |
| | 25.0% | 0.94 | 0.42 |
| PEG 300 | 5.0% | 0.39 | 0.27 |
| | 10.0% | 0.64 | 0.34 |
| | 25.0% | 1.61 | 0.87 |
| PEG 400 | 5.0% | 0.39 | 0.21 |
| | 10.0% | 0.63 | 0.45 |
| | 25.0% | 1.47 | 0.93 |
| Glycerol | 5.0% | 0.18 | 0.20 |
| | 10.0% | 0.22 | 0.19 |
| | 25.0% | 0.38 | 0.37 |

Based on these results, a formulation based on conventional excipients could not be identified.

### Comparative Example 2a

The following excipients were evaluated for their use as preservatives in oral solutions: propionic acid; bronopol; phenol; chlorobutol; benzalkonium chloride; thiomerosal; benzyl alcohol; and parabens. Oral Permissible Daily Exposure (PDE) values of 9.3-22 mg/day for propionic acid, 0.19-0.46 mg/day for bronopol, 0.036-0.084 mg/day for chlorbutol, 0.038-0.091 mg/day for phenol, 2.8 mg/kg/day for methylparaben and 2.0 mg/kg/day for Propylparaben were calculated for paediatric patient populations (new-borns, infants and toddlers). From a toxicological perspective, propionic acid, benzoic acid and parabens were investigated as preservatives for branaplam oral solution. The HP-b-CD concentration was reduced to 7.5% (w/v) and 10% (w/v) in order to minimize the concentration of free HP-b-CD available to interact with the preservatives. Lower concentrations of HP-b-CD showed precipitation after storage in the fridge and therefore are not recommended. The branaplam solution was prepared as described for Examples 1a-18a, followed by adding the intended preservatives with the specified concentrations.

TABLE 6a
Branaplam multiple dose formulation development: AET results.
Composition [% w/v]

| Formulation | F1 | F2 | F3 | F4 | F5 | F6 | F7 | F8 | F9 | F10 | F11 |
|---|---|---|---|---|---|---|---|---|---|---|---|
| Branaplam HCI | 0.383 | 0.383 | 0.383 | 0.383 | 0.383 | 0.383 | 0.383 | 0.383 | 0.383 | 0.383 | 0.383 |
| salt¹>²> | | | | | | | | | | | |
| HP-b- | 7.5 | 7.5 | 7.5 | 7.5 | 10.0 | 10.0 | 7.5 | 7.5 | 7.5 | 7.5 | 7.5 |
| Cyclodextrin | | | | | | | | | | | |
| Benzoic acid | 0.05 | 0.1 | 0.2 | | 0.1 | 0.2 | 0.5 | | 0.5 | | |
| Propionic acid | | | | 0.2 | | | | | | 0.5 | 0.5 |
| Metyl paraben | | | | | | | | 0.3 | 0.3 | | 0.3 |
| Propyl paraben | | | | | | | | 0.2 | 0.2 | | 0.2 |
| Final pH | pH 4.0 ± 0.2 | | | | | | | | | | |
| Holding time | <3 | <3 | <3 | <3 | <3 | <3 | n.a. | n.a. | n.a. | n.a. | n.a. |
| [days] | | | | | | | | | | | |
| AET [Pass/fail] | n.a. ³⁾ | n.a. ³⁾ | n.a. ³⁾ | n.a. ³⁾ | n.a. ³⁾ | n.a. ³⁾ | n.a. ⁴⁾ | Fail | n.a. ⁴⁾ | Pass | Pass |

¹⁾Branaplam weight adjustments were performed for drug substance content ≤99.5% and less amount of water for DS compensation.
²⁾0.383% Branaplam HCl salt corresponds to 3.5 mg/mL branaplam (salt/free form ratio on anhydrous basis 1.093)
³⁾ AET Not performed as holding time was <3 days
⁴⁾ AET Not performed as precipitation was observed, when stored at 2-8° C.

Table 6a shows the Antimicrobiologic Effectiveness Testing (AET) results of the tested formulations, performed in accordance with USP “<51> ANTIMICROBIAL EFFECTIVENESS TESTING”, the version valid as of May 1, 2012. Multidose formulations must comply with the AET testing. Precipitation was observed when the HP-b-CD concentration was reduced to 7.5% (w/v) and in the presence of benzoic acid. Formulations with 0.2% (w/v) propyl paraben and 0.3% (w/v) methyl paraben and 7.5% (w/v) HP-b-CD failed AET testing. From the tested formulations, only the formulations with propionic acid with or without parabens met the AET specification. However, propionic acid is volatile and with an aversive smell; consequently, its use for paediatric oral solution is not recommended.

Based on these results, a formulation to support multiple dosing could not be identified.

### Example 19a

Taste assessment of branaplam oral solutions (as in Example 18a) with and without sweeteners and flavours was performed in human volunteers. Table 7a shows the level of participants' reported perception of an aversive aftertaste and willingness to take the sample as a medicine for chronic use and the Visual Analogue Scale (VAS) using the scale 0 “Pleasant” and 100 “aversive”. The formulation without any taste-masking or flavouring excipients rated near the midpoint on the continuous VAS scale. The taste of the drug was described as “bitter” and “aversive”, with a particular problem with aftertaste. Addition of 0.05% sucralose and 0.1% vanilla (w/v) was most effective at taste-masking, and most favoured by the participants with 11 out 12 participants willing to take the formulation in comparison with only 5 willing to take the formulation without any taste-masking or flavouring excipients. The formulation containing 0.05% sucralose and 0.1% vanilla was rated as significantly less aversive (VAS=12.5) compared to the formulation without any taste-masking or flavouring excipients (VAS=54), and no participants rated it negatively on the 5-point categorical facial scale (Table 8a). All other formulation combinations that were tested were not as effective at masking the aversive taste and aftertaste of the drug.

TABLE 7a
Branaplam aversive aftertaste.

| | | | | | | VAS |
|---|---|---|---|---|---|---|
| | Aversive aftertaste | | | | | overall ratings |
| | Yes, a | Yes-a | | Willing | | (0 “pleasant”, |
| | strong | slight | | to take | | 100 |
| Formulations | aftertaste | aftertaste | No | Yes | No | “aversive”) |
| 17.5% Cyclodextrin, pH | 4 | 6 | 2 | 5 | 7 | 54 |
| 4.0 | | | | | | |
| 17.5% Cyclodextrin, 0.05% | 1 | 9 | 2 | 10 | 2 | 29.5 |
| Sucralose pH 4.0 | | | | | | |
| 17.5% Cyclodextrin, 0.05% | 2 | 7 | 3 | 11 | 1 | 12.5 |
| Sucralose, 0.1% Vanilla | | | | | | |
| pH 4.0 | | | | | | |
| 17.5% Cyclodextrin, 0.05% | 2 | 8 | 2 | 9 | 3 | 19.5 |
| Sucralose, 0.05% sodium | | | | | | |
| sacharine pH 4.0 | | | | | | |
| 17.5% Cyclodextrin, 0.05% | 4 | 7 | 1 | 6 | 6 | 35 |
| Sucralose, 0.05% sodium | | | | | | |
| sacharine, 0.1% Vanilla pH | | | | | | |
| 4.0 | | | | | | |

The cyclodextrin in the table refers to HP-b-CD, as in Example 18a.
The concentrations expressed in percentage for sucralose and vanillin refer to % w/v.

TABLE 8a
Categorical Scale Raw Data.

| | 1 | 2 | 3 | 4 | 5 |
|---|---|---|---|---|---|
| Formulation Samples | [symbol] | [symbol] | [symbol] | [symbol] | [symbol] |
| 17.5% Cyclodextrin, pH 4.0 | 0 | 2 | 3 | 7 | 0 |
| 17.5% Cyclodextrin, 0.05% | 3 | 5 | 3 | 1 | 0 |
| Sucralose pH 4.0 | | | | | |
| 17.5% Cyclodextrin, 0.05% | 4 | 6 | 2 | 0 | 0 |
| Sucralose, 0.1% Vanilla pH | | | | | |
| 4.0 | | | | | |
| 17.5% Cyclodextrin, 0.05% | 4 | 4 | 3 | 1 | 0 |
| Sucralose, 0.05% sodium | | | | | |
| sacharine pH 4.0 | | | | | |
| 17.5% Cyclodextrin, 0.05% | 2 | 4 | 2 | 4 | 0 |
| Sucralose, 0.05% sodium | | | | | |
| sacharine, 0.1% Vanilla pH | | | | | |
| 4.0 | | | | | |

The cyclodextrin in the table refers to HP-b-CD, as in Example 18a.
The concentrations expressed in percentage for sucralose and vanillin refer to % w/v.

The procedure for preparing the above-mentioned solutions was as follows. The required amount of 2-hydroxypropyl-beta-cyclodextrin was dissolved in 80% volume of target water (i.e. final intended volume) and stirred for 30 minutes. The required amount of branaplam was then added to said solution, under stirring, at room temperature. The solution was stirred for 45 minutes after the addition was completed or for longer until a particle-free solution was obtained. Initial pH adjustment was performed using NaOH 0.1M or HCL 0.1M to reach the intended pH (±0.25). The required amount of sucralose was added to the solution under stirring, at room temperature, and stirring was continued for at least 10 minutes after the addition was completed. The required amount of vanilla was added to the solution under stirring, at room temperature, and stirring was continued for at least 10 minutes after the addition was completed. The required volume of water was added to solution to reach the final intended volume and stirred for at least 10 minutes after the addition was completed. Final pH adjustment was performed using NaOH 0.1 M or HCL 0.1 M to reach the intended pH.

### Examples 20a-24a

Examples 20a-24a describe some of the preferred embodiments of the present invention. The details of oral formulations of branaplam as in said examples are given in Tables 9a and 10a.

TABLE 9a
Oral formulation of branaplam comprising
up to 17.5% (w/v) Captisol ®.

| | Examples | | | |
|---|---|---|---|---|
| | 20a | 21a | 22a | 23a |
| Ingredients | Amount | | | |
| Captisol ® (% w/v) | 2.5 | 7.5 | 12.5 | 17.5 |
| Branaplam monohydrochloride salt (mg/ml) | 4.5 | 9.3 | 12.7 | 15.0 |

| | |
|---|---|
| Hydrochloride/acetic/phosphoric/lactic/ | q.s. to pH 4 |
| tartaric/citric acid | |
| Sodium hydroxide | |
| Water | q.s. |
| pH adjusted to | 4.0 |

TABLE 10a
Oral formulation of branaplam according to Example 24a.

| Ingredients | Amount | |
|---|---|---|
| Branaplam monohydrochloride salt | 3.826 | mg/ml |
| Captisol ® | 2.5 | percent (w/v) |

| | |
|---|---|
| Hydrochloride acid | q.s. to pH 4 |
| Sodium hydroxide | |
| Water | q.s. |
| pH adjusted to | 4 |

Procedure:

The required amount of Captisol® was dissolved in 80% volume of target water (i.e. final intended volume) and stirred for 30 minutes. The required amount of branaplam monohydrochloride salt was then added to said solution, under stirring, at room temperature.

The solution was stirred for 45 minutes after the addition was completed or for longer until a particle-free solution was obtained. Initial pH adjustment was performed using NaOH 0.1M or HCl 0.1M to reach the intended pH (±0.25). The required volume of water was added to solution to reach the final intended volume and stirred for at least 10 minutes after the addition was completed. Final pH adjustment was performed using NaOH 0.1 M or HCL 0.1 M to reach the intended pH.

### Example 25a

This example provides and exemplary method for the preparation of preservative-free formulations of branaplam.

Different branaplam solutions were prepared according to the procedure as described for examples 1a-24a, each one in a total volume of 40 litres. Each solution was then filtered (pre-filtering) through a 0.45 μm filter. The first 20 mL of the bulk solution through the filter was discarded to confirm the flushing volume for the filter cartridge. The solution was then filtered (sterile filtering) through a 0.22 μm filter. The first 500 mL of the bulk solution through the filter was discarded to confirm the flushing volume for the filter cartridge. The filtered solution was then filled into amber glass vials (6 ml per vial) and closed with lyophilizer stopper and tearable aluminium cap. Optionally, the vials have child-resistant/temper evident closure system. The process for preparation of a branaplam formulation using HP-b-CD is illustrated in FIG. 1. The same process applies when sulfobutylether β-cyclodextrin sodium salt (e.g. Captisol©) is used instead of HP-b-CD.

TABLE 11a
Oral formulation of branaplam with reduced cyclodextrin

| Ingredients | Amount | |
|---|---|---|
| Branaplam monohydrochloride salt | 3.826 | mg/ml |
| 2-hydroxypropyl-beta-cyclodextrin | 10.0 | percent (w/v) |

| | |
|---|---|
| Hydrochloride acid | q.s. to pH 4 |
| Sodium hydroxide | |
| Water | q.s. |
| pH adjusted to | 4 |

Clinical Trial: An Open-Label Multi-Part First-In-Human Proof of Concept Study of Oral Branaplam in Infants with Type I Spinal Muscular Atrophy.
Part 1: The aim of part one of this study was to determine the safety and tolerability of ascending weekly doses and to estimate the maximum tolerated dose (MTD) of oral/enteral branaplam in infants with Type 1 SMA. All patients had exactly 2 copies of the SMN2 gene.
In part one of the study, patients were dosed once weekly with branaplam. The branaplam doses were escalated in subsequent cohorts until MTD was determined or when PK results confirmed that the MTD could not be reached due to a potential pharmacokinetic exposure plateau at higher doses. A decision to dose escalate the next cohort was made after safety data had been collected for 14 days following the first dose. Patients completing 13 weeks of treatment were considered to have completed the study. The starting dose was 6 mg/m² (approximately 0.3125 mg/kg). Subsequent doses were 12 mg/m², 24 mg/m², 48 mg/m² and 60 mg/m² (approximately 0.625 mg/kg, 1.25 mg/kg, 2.5 mg/kg and 3.125 mg/kg, respectively). Each cohort had 2-3 patients. All doses are of branaplam in free form. 14 patients were enrolled in Part 1; 13 patients were exposed to branaplam. The duration of exposure ranged from 4-33 months, 7 patients remain in the study. Six of the 7 patients are receiving 60 mg/m², 1 patient is receiving 48 mg/m². No dose limiting toxicity was observed and exposures (AUC) were comparable for 48 and 60 mg/m².

Preliminary Safety Results
AEs/SAEs: 455 AEs were reported in the 13 patients; the vast majority were attributed to the underlying disease. Seventy-nine SAEs occurred associated with 59 hospitalizations of which 39 of the 59 hospitalizations were for respiratory events or infections.
Deaths: A total of five patients have died, all from ventilatory failure due to their underlying disease. Two patients died shortly after a dose reduction to 6 mg/m² that was implemented as an urgent safety measure following findings of nerve degeneration in the 52-week chronic juvenile dog study.
This led to motor stabilization and, in several cases, return of some motor function in patients.

Preliminary Efficacy Results
CHOP INTEND motor scale score (measures muscle strength in very weak infants): A progressive and substantial increase of CHOP INTEND scores overtime was observed in 7 of the 12 evaluable patients at 13 weeks of treatment; no significant decrease was observed in any patient. Eight patients have reached a CHOP INTEND score of >36, exceeding the results seen in historical control studies.
Hammersmith Infant Neurological Examination—Section 2 (HINE, assesses 8 categories of infant motor milestones that are achieved through 18 months of age in typically developing infants): Of the 11 patients evaluated, one achieved independent sitting, a milestone never reported in natural history studies in Type 1 SMA (lack of independent sitting is the definition of Type 1 SMA).
Clinical status: Branaplam-treated patients do not follow the normal disease course for Type 1 SMA patients.
   Feeding support: The median age for feeding support in Type 1 SMA patients is around 8 months of age (natural history studies). Ten treated patients did not receive any feeding support at this age, six did not receive feeding support after more than one year of treatment and five are not receiving feeding support after more than two years of treatment.
   Death or permanent ventilation: The median age to reach this endpoint is 13.5 months (natural history studies). The majority of treated patients have not met this endpoint at this time. Four patients, treated for more than two years, do not receive any ventilatory support with BiPAP.
Part 2: The aim of part two of this study is to evaluate the long term safety and tolerability of 2 doses of branaplam administered weekly for 52 weeks in patients with Type 1 SMA. Part 2 of the study will enroll patients into 2 cohorts: cohort 1 at a 0.625 mg/kg dose and cohort 2 at a 2.5 mg/kg dose. The selected dose levels of 0.625 mg/kg and 2.5 mg/kg are based on all safety data from Part 1, as well as, all data from chronic juvenile toxicity studies available at the time of initiation of Part 2. Six to 10 patients will be enrolled in cohorts 1 and 2. A total of a minimum of 12 and maximum of 20 patients will be enrolled and treated for 52 weeks.
Starting dose: The dose of 0.625 mg/kg (corresponding to 12 mg/m²) is chosen as a starting dose in Part 2 based on safety and preliminary efficacy data collected in Part 1 of the study. Indeed, following an USM treatment with branaplam continued but at a reduced dose of 6 mg/m² (0.3125 mg/kg) for all patients who had completed the initial 13 weeks of treatment. This dose was predicted to be efficacious based upon the preliminary clinical (CHOP INTEND) response of patients in Cohort 1. However, following the branaplam dose reduction to 6 mg/m² (0.3125 mg/kg), safety events including decrease of motor skills, generalized motor weakness and increased respiratory muscle weakness were reported. While a mechanistic relation between these clinical observations and the preclinical nerve fiber degeneration following branaplam treatment cannot be completely excluded, due to the almost synchronous temporal association of the events with the decrease in dose and the similar course of the events in the 7 patients, it is most likely that branaplam was benefitting the patients and that the lower dose of branaplam is less effective. For that reason and also given the preliminary efficacy data collected in patients treated with branaplam at 12 mg/m² (0.625 mg/kg) it was decided to take this dose as a starting dose in Part 2.
Second dose: The dose of 2.5 mg/kg (corresponding to 48 mg/m²) is selected as the second dose in Part 2 being 4-fold higher than the starting dose of 0.625 mg/kg (corresponding to 12 mg/m²). The difference between 0.625 mg/kg and 2.5 mg/kg is considered to be sufficient to ensure appropriate separation in terms of systemic branaplam exposure and potentially also of efficacy endpoints. An increase of the selected second dose of 2.5 mg/kg and 3.125 mg/kg (corresponding to 48 mg/m² to 60 mg/m²) was not further considered as the dose difference is only 1.25-fold and exposure overlap was clearly observed in Part 1.

1. A pharmaceutical composition comprising
A) The compound of formula (I) 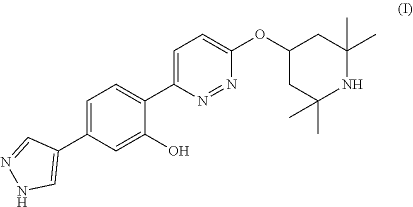 [SMILES: CC1(C)CC(Oc2ccc(-c3ccc(-c4cn[nH]c4)cc3O)nn2)CC(C)(C)N1]
or a pharmaceutically acceptable salt thereof, and
B) a pharmaceutically acceptable cyclodextrin or combination of pharmaceutically acceptable cyclodextrins.

2. The pharmaceutical composition of claim 1, wherein the compound of formula (I) is in its hydrochloride salt form.

3. The pharmaceutical composition of claim 1, wherein the cyclodextrin is a beta-cyclodextrin, for example selected from the group consisting of 2-hydroxypropyl-beta-cyclodextrin, sulfobutylether-beta-cyclodextrin, beta-cyclodextrin, methyl-beta-cyclodextrin, hydroxyethyl-beta-cyclodextrin, ethyl-beta-cyclodextrin, butyl-beta-cyclodextrin, Succinyl-(2-hydroxypropyl)-beta-cyclodextrin, heptakis(2,3,6-tri-O-methyl)-beta-cyclodextrin, heptakis(2,3,6-tri-O-benzoyl)-beta-cyclodextrin, beta-cyclodextrin phosphate sodium salt, beta-cyclodextrin sulphate sodium salt, triacetyl-beta-cyclodextrin, heptakis(6-O-sulfo)-beta-cyclodextrin heptasodium salt, carboxymethyl-beta-cyclodextrin sodium salt, sulfobutylether-beta-cyclodextrin sodium salt, and 6-O-p-toluenesulfonyl-beta-cyclodextrin.

4. The pharmaceutical composition according to claim 1, wherein the composition is a liquid composition.

5. The pharmaceutical composition according to claim 1, wherein cyclodextrin (B) is 2-hydroxypropyl-beta-cyclodextrin.

6. The pharmaceutical composition according to claim 1, wherein cyclodextrin (B) is sulfobutylether-beta-cyclodextrin.

7. The pharmaceutical composition according to claim 1, wherein the concentration of the compound of formula I or any pharmaceutically acceptable salt thereof is in the range of about 1 mg/ml to about 30 mg/ml, for example in the range of about 3 mg/ml to about 10 mg/ml.

8. The pharmaceutical composition according to claim 1, wherein the cyclodextrin is present in a concentration in the range of 2 percent to 25 percent (w/v).

9. The pharmaceutical composition according to claim 1, wherein the pH of the composition is in the range of 3.5 to 9, for example about 4.

10. The pharmaceutical composition according to claim 1, said composition comprising:
A) the compound of formula I or a pharmaceutically acceptable salt thereof in a concentration of 1 mg/ml to 30 mg/ml,
B) 2-hydroxypropyl-beta-cyclodextrin in a concentration in the range of 2 percent to 25 percent (w/v), for example in the range of 10 percent to 20 percent (w/v).
and wherein the pH of the composition is about 4.0.

11. The pharmaceutical composition according to claim 1, wherein the composition further comprises at least one taste-masking agent, for example sucralose.

12. The pharmaceutical composition according to claim 1, wherein the composition further comprises at least one flavouring agent, for example vanilla.

13. The pharmaceutical composition according to claim 1, said composition comprising:
a) the hydrochloride salt of the compound of formula (I) in a concentration of 3.5 mg/ml,
b) 2-hydroxypropyl-beta-cyclodextrin in a concentration of 17.5 percent (w/v),
c) sucralose in a concentration of 0.05 percent (w/v),
d) vanilla in a concentration of 0.1 percent (w/v)
e) water
and wherein the pH of the composition is about 4.0 or higher.

14. The pharmaceutical composition according to claim 1, said composition comprising:
a) the compound of formula I, or a pharmaceutically acceptable salt thereof [e.g. the hydrochloride salt of the compound of formula (I)] in a concentration of 1 mg/ml to 30 mg/ml, for example 3.5 mg/ml,
b) a pharmaceutically acceptable cyclodextrin (e.g., 2-hydroxypropyl-beta-cyclodextrin) in a concentration of 10.0 percent (w/v),
c) at least one taste-masking agent, for example sucralose, in a concentration of from 0.05% to 0.5% (w/v), for example 0.05% (w/v),
d) optionally at least one flavouring agent, for example vanilla, in a concentration of from 0.05% to 0.2% (w/v), for example 0.1% (w/v).
e) water
and wherein the pH of the composition is about 4.0 or higher (e.g., about 4 to about 7).

15. The pharmaceutical composition according to claim 1, wherein the composition is substantially free of preservatives.

16.-17. (canceled)

18. A method to treat, prevent or ameliorate a SMN-deficiency-related condition, for example Spinal Muscular Atrophy (SMA), comprising administering to a subject in need thereof an effective amount of a composition according to claim 1.

19. The method of claim 18 wherein the composition is administered at a dose of about 0.625 mg/kg to about 3.125 mg/kg of branaplam, in free form or in the form of a pharmaceutically acceptable salt, per weight of subject.

20. The method of claim 18, wherein the composition is administered orally.
